(19) United States
(12) Patent Application Publication
(10) Pub. No.: US 2020/0085971 A1
(43) Pub. Date: Mar. 19, 2020

(54) METHODS AND COMPOSITIONS FOR MODULATION OF IMMUNE CELLS

(71) Applicant: QT Holdings Corp, Woburn, MA (US)

(72) Inventors: Sean H. Kevlahan, North Reading, MA (US); Andrew Ball, Georgetown, MA (US); Guokui Qin, Somerville, MA (US); Steven B. Wells, Allston, MA (US); Nithya Jothi Jesuraj, Woburn, MA (US); Julie M. Cole, Wakefield, MA (US)

(21) Appl. No.: 16/494,197

(22) PCT Filed: Mar. 20, 2018
(86) PCT No.: PCT/US18/23318

§ 371 Date: Sep. 13, 2019

Related U.S. Application Data
(60) Provisional application No. 62/473,730, filed on Mar. 20, 2017.

(51) Int. Cl. A61K 47/69; A61K 47/34; A61K 47/54; C07K 16/28; A61K 47/18; A61K 47/10; A61K 35/17; C12N 5/00; C12N 5/0783

(52) U.S. Cl. CPC A61K 47/6903; A61K 47/34; A61K 47/555; C07K 16/28; A61K 47/183; A61K 47/10; C12N 2533/40; A61K 35/17; C12N 5/0068; C12N 5/0636; C12N 2533/54; C12N 2533/74; C12N 2533/80; A61K 47/6901

(57) ABSTRACT

The invention features a hydrogel complex that can bind to and modulate a desired immune cell, e.g., T cell, population. In certain embodiments, the complex can be dissolved, and thus dissociated from its targeted cell, representing a safe and efficient approach for processing immune cells, e.g., T cells for clinical use. The invention also provides methods and apparatus for synthesizing hydrogel complexes, as well as methods of using the complexes to generate expanded immune cell, e.g., T cell, populations as part of adoptive immune cell, e.g., T cell, therapy systems.

### BACKGROUND

A rapidly emerging field of therapeutic research involves the transplantation of autologous or allogenic immune cells into patients to treat cancer and other diseases. Many types of immune cells are being evaluated for this purpose, including lymphocytes, NK cells, NKT cells, CIK cells, dendritic cells, stem cell-derived immune cells, and other immune cell types and subtypes. Thus far, T lymphocyte-based therapies have advanced furthest clinically, although other immune cell types have shown considerable therapeutic promise in preclinical studies. T lymphocytes isolated from whole blood are utilized in a wide variety of in vitro, in vivo, and clinical research and therapeutic applications. Examples include studies of immune response, T cell receptor signaling, cytokine release and gene expression profiling. Perhaps most significantly, isolation and subsequent ex vivo engineering of T lymphocytes for subsequent transplantation into clinical patients is showing tremendous promise as a novel cancer therapy. The main approaches to this are engineering of T cells to express either chimeric antigen receptors (CAR) or T cell receptors (TCR). In both approaches, T cells are isolated from whole blood, activated and expanded ex vivo, and subsequently infused into human subjects.

Although both polyclonal and antigen-specific T cells can be readily isolated from whole blood, their numbers are limited. Accordingly, protocols that activate and promote ex vivo expansion of T cells are widely used. Such ex vivo manipulations, however, can potentially reduce T cell viability, proliferation, and survival after infusion. Thus, the choice of methods used for T cell activation has important implications for clinical efficacy.

It is well-established that, in vivo, activation of T cells is dependent on two signals; engagement of the T cell receptor with antigen (signal 1) and ligation of a costimulatory molecule (signal 2). Both are required for an effective immune response. Ex vivo, T cell activation is most commonly induced by exposing the T cells to antibodies directed against the T cell surface markers CD3 and CD28 to engage the T cell receptor and deliver a costimulatory signal simultaneously.

There are significant disadvantages of conventional ex vivo T cell activation protocols, which use magnetic beads, resulting from the presence of residual magnetic beads attached to the cells. These may negatively affect both function and viability. Pre-clinical clinical applications require cells are that are free from contaminating particles, while retaining high viability. For example, June et al. (Pilot study of redirected autologous T cells engineered to contain humanized anti-CD19 in patients with relapsed or refractory CD19⁺ leukemia and lymphoma previously treated with cell therapy (2015) ClinicalTrials.gov) specified final product release criteria in the IND included the specifications that the number of anti-CD3/anti-CD28-coated paramagnetic beads should not exceed 100 per 3×10⁶ cells and that cell viability should be greater than 70%. However, minimizing the number of beads represents a formidable obstacle in the clinical translation of such therapies, as most antibody-coated magnetic-bead based products lack the ability to release bound cells readily from capture molecules in a manner that does not alter the viability and phenotype of the isolated cells.

Given the significant interest in, and rapid expansion of, T cell engineering-based cancer therapies, there is a significant need for improved T cell expansion and harvesting methods that overcome the above limitations of existing approaches, particularly for downstream clinical applications. Specifically, there is a substantial need for technologies to enable ex vivo cell expansion protocols to meet clinical specifications, to expand T and other immune cells consistently and reproducibly, to preserve cell viability and function, and to be applicable to different cell sources and expansion agents.

### SUMMARY OF THE INVENTION

The present invention features a biocompatible hydrogel complex capable of binding to, activating, and expanding immune cell, e.g., T cells. In certain embodiments, the hydrogel complex can be dissolved, e.g., simply by reducing cation concentration, e.g., by introducing a chelating agent, enabling efficient production of large numbers of T cells for adoptive transfer systems and other uses of immune cells. Also provided herein are methods for producing hydrogel complexes and methods of generating expanded and/or activated immune cell, e.g., T cell, populations using the hydrogel complexes of the invention.

In one aspect, the invention features a particle including a complex including a hydrogel and a binding moiety, wherein the hydrogel includes a polymer; and the binding moiety is configured to bind a cell surface component of an immune cell.

In some embodiments, the polymer includes a natural polymer. Exemplary natural polymers are alginate, agarose, carrageenan, chitosan, dextran, carboxymethylcellulose, heparin, hyaluronic acid, polyamino acids, collagen, gelatin, fibrin, fibrous protein-based biopolymers, and any combination thereof. In other embodiments, the polymer includes a synthetic polymer. Exemplary synthetic polymers are alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene (e.g., expanded), ethylene vinyl alcohol copolymer, poly(hexamethylene diisocyanate), poly(ethylene) (e.g., high density, low density, or ultrahigh molecular weight), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

The polymer may also be a copolymer, e.g., a copolymer of a natural polymer, e.g., alginate, with a synthetic polymer, e.g., PEG or PMOXA. Alternatively, the polymer is not a copolymer of alginate and PEG. Other examples include a copolymer of hyaluronic acid with PEG or dextran with PEG. In other embodiments, the polymer is not a copolymer of alginate with a fluoropolymer or a silicone.

In some embodiments, the cell surface component is CD2, CD3, CD19, CD24, CD27, CD28, CD31, CD34, CD45, CD46, CD80, CD86, CD133, CD134, CD135, CD137, CD160, CD335, CD337, CD40L, ICOS, GITR, HVEM, Galtectin 9, TIM-1, LFA-1, PD-L1, PD-L2, B7-H3, B7-H4, ILT3, ILT4, CDTL-4, PD-1, BTLA, MHC-I, MHC-II, Delta-like ligand (e.g., DLL-Fc, DLL-1, or DLL-4), WNT3, stem cell factor, or thrombopoietin. In some embodiments, the cell surface component is CD2, CD3, CD27, CD28, CD46, CD80, CD86, CD134, CD137, CD160, CD40L, ICOS, GITR, HVEM, Galtectin 9, TIM-1, LFA-1, PD-L1, PD-L2, B7-H3, B7-H4, ILT3, ILT4, CDTL-4, PD-1, BTLA, MHC-I, or MHC-II. Alternatively, the cell surface component is not CD2, CD3, CD27, CD28, CD46, CD80, CD86, CD134, CD137, CD160, CD40L, ICOS, GITR, HVEM, Galtectin 9, TIM-1, LFA-1, PD-L1, PD-L2, B7-H3, B7-H4, ILT3, ILT4, CDTL-4, PD-1, BTLA, MHC-I, or MHC-II, in particular when the polymer is a copolymer of alginate, e.g., a copolymer of alginate and PEG. In other embodiments, the cell surface component is CD24, CD31, CD34, or CD45. Alternatively, the cell surface component is not CD24, CD31, CD34, or CD45, in particular when the polymer is a copolymer of alginate, e.g., a copolymer of alginate and PEG. In other embodiments, the cell surface component is CD19, CD34, CD45, CD133, CD135, CD335, CD337, DLL-Fc, DLL-1, or DLL-4, WNT3, stem cell factor, or thrombopoietin, e.g., CD19, CD133, CD135, CD335, CD337, Delta-like ligand (e.g., DLL-Fc, DLL-1, or DLL-4), WNT3, stem cell factor, or thrombopoietin.

In some embodiments, the surface of the particle includes at least one binding moiety per square μm (e.g., at least 1 binding moiety per square μm, at least 2 binding moieties per square μm, at least 3 binding moieties per square μm, at least 4 binding moieties per square μm, at least 5 binding moieties per square μm, at least 10 binding moieties per square μm, at least 20 binding moieties per square μm, at least 30 binding moieties per square μm, at least 40 binding moieties per square μm, or at least 50 binding moieties per square μm). In some instances, one or more of the binding moieties is an antibody or antigen-binding fragment thereof.

The particle may include one, two, three, or more distinct binding moieties.

A binding moiety can be an antibody or antigen-binding fragment thereof. For example, a binding moiety is a monoclonal antibody or antigen-binding fragment thereof, a Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′) 2) molecule, a domain antibody, a diabody, a triabody, an affibody, a domain antibody, a SMIP, a nanobody, a Fv fragment, a Fab fragment, a F(ab′) 2 molecule, or a tandem scFv (taFv) fragment. The antibody or antigen binding fragment thereof is, for example, anti-CD2, anti-CD3, anti-CD19, anti-CD24, anti-CD27, anti-CD28, anti-CD31, anti-CD34, anti-CD45, anti-CD46, anti-CD80, anti-CD86, anti-CD133, anti-CD134, anti-CD135, anti-CD137, anti-CD160, anti-CD335, anti-CD337, anti-CD40L, anti-ICOS, anti-GITR, anti-HVEM, anti-Galtectin 9, anti-TIM-1, anti-LFA-1, anti-PD-L1, anti-PD-L2, anti-B7-H3, anti-B7-H4, anti-ILT3, anti-ILT4, anti-CDTL-4, anti-PD-1, anti-BTLA, anti-MHC-I, anti-MHC-II, anti-Delta-like ligand (e.g., anti-DLL-Fc, anti-DLL-1, or anti-DLL-4), anti-WNT3, anti-stem cell factor, or anti-thrombopoietin. In some embodiments, the antibody or antigen-binding fragment thereof is anti-CD2, anti-CD3, anti-CD27, anti-CD28, anti-CD46, anti-CD80, anti-CD86, anti-CD134, anti-CD137, anti-CD160, anti-CD40L, anti-ICOS, anti-GITR, anti-HVEM, anti-Galtectin 9, anti-TIM-1, anti-LFA-1, anti-PD-L1, anti-PD-L2, anti-B7-H3, anti-B7-H4, anti-ILT3, anti-ILT4, anti-CDTL-4, anti-PD-1, anti-BTLA, anti-MHC-I, or anti-MHC-II. Alternatively, the antibody or antigen-binding fragment thereof is not anti-CD2, anti-CD3, anti-CD27, anti-CD28, anti-CD46, anti-CD80, anti-CD86, anti-CD134, anti-CD137, anti-CD160, anti-CD40L, anti-ICOS, anti-GITR, anti-HVEM, anti-Galtectin 9, anti-TIM-1, anti-LFA-1, anti-PD-L1, anti-PD-L2, anti-B7-H3, anti-B7-H4, anti-ILT3, anti-ILT4, anti-CDTL-4, anti-PD-1, anti-BTLA, anti-MHC-I, or anti-MHC-II, in particular when the polymer is a copolymer of alginate, e.g., a copolymer of alginate and PEG. In some embodiments, the antibody or antigen-binding fragment thereof is anti-CD24, anti-CD31, anti-CD34, or anti-CD45. Alternatively, the antibody or antigen-binding fragment thereof is not anti-CD24, anti-CD31, anti-CD34, or anti-CD45, in particular when the polymer is a copolymer of alginate, e.g., a copolymer of alginate and PEG. In other embodiments, the antibody or antigen-binding fragment thereof is anti-CD19, anti-CD34, anti-CD45, anti-CD133, anti-CD335, anti-CD337, anti-DLL-Fc, anti-DLL-1, anti-DLL-4 anti-WNT3, anti-stem cell factor, or anti-thrombopoietin, e.g., anti-CD19, anti-CD133, anti-CD335, anti-CD337, anti-Delta-like ligand (e.g., anti-DLL-Fc, anti-DLL-1, or anti-DLL-4), anti-WNT3, anti-stem cell factor, or anti-thrombopoietin.

The binding moiety may be a signal 1 stimulus (e.g., anti-CD3) or a signal 2 stimulus (e.g., anti-CD28). In complexes having both a signal 1 stimulus (e.g., anti-CD3) and a signal 2 stimulus (e.g., anti-CD28), the molar ratio of the signal 1 stimulus and the signal 2 stimulus can be between about 1:100 and about 100:1 (e.g., from 1:80 to 80:1, from 1:60 to 60:1, from 1:50 to 50:1, from 1:40 to 40:1, from 1:30 to 30:1, from 1:20 to 20:1, from 1:10 to 10:1, from 1:5 to 5:1, from 1:2 to 2:1, or about 1:1). In some embodiments, the signal 1 stimulus is antigen-specific.

Alternatively, a complex may include three or more types of binding moieties. For example, in some embodiments, the complex includes a signal 1 stimulus (e.g., anti-CD3), a signal 2 stimulus (e.g., anti-CD28), and an additional stimulus, such as an activating stimulus, a suppressive stimulus, or a polarizing stimulus, such as a cytokine (e.g., a surface-bound cytokine, e.g., a trans-presented interleukin).

In certain embodiments, the binding moiety is a cytokine. For example, the cytokine is IL-1, IL-2, IL-3, IL-6, IL-7, IL-12, IL-15, IL-18, IL-21, TNF-α, or IFN-γ. In other embodiments, the binding moiety is chemokine (C-X-C motif) ligand 12 or low-density lipoprotein.

The immune cell is, for example, naïve and memory T cells, T helper cell, regulatory T cell, NK cell, NK T cell, CIK cell, TIL cell, HS cell (undifferentiated and differentiated), MS cell (undifferentiated and differentiated), iPS cell (undifferentiated and differentiated), B cell, macrophage, dendritic cell, neutrophil, stromal cell, and ES cell (undifferentiated and differentiated). In certain embodiments, the immune cell is an NK cell, CIK cell, TIL cell, HS cell (undifferentiated and differentiated), MS cell (undifferentiated and differentiated), iPS cell (undifferentiated and differentiated), or ES cell (undifferentiated and differentiated). In other embodiments, the immune cell is a T cell, e.g., a naïve or memory T cell, T helper cell, regulatory T cell, Natural Killer T cell, or Cytotoxic T Lymphocyte, or a B cell, macrophage, dendritic cell, neutrophil, or stromal cell.

In some embodiments, the polymer can change from a solid matrix into a solution or suspension, e.g., in response to a sufficient decrease of cationic concentration in the environment of the polymer, change in temperature, change in pH, due to hydrolysis, oxidation, enzymatic degradation, physical degradation, or other mechanism. For example, the decrease in the cationic concentration in the environment of the polymer can be caused by the presence of EDTA, EGTA, sodium citrate, BAPTA, crown ether, cryptand, phenanthroline sulfonate, dipyridyl sulfonate, dioxane, DME, diglyme, or triglyme. In some embodiments, the cation is Li⁺, Mg²⁺, Ca²⁺, Sr²⁺, Ba²⁺, Zn²⁺, Cu²⁺, or Al³⁺. In some embodiments, the particle completely liquefies, e.g., in response to a sufficient decrease of cationic concentration in the environment of the particle, e.g., the particle does not further include a separation unit, such as a magnetic bead substrate or magnetic particles.

In some embodiments, the hydrogel has an elastic modulus of less than 1 gigapascal (GPa), e.g., 0.8 GPa, 0.6 GPa, 0.4 GPa, 0.2 GPa, 0.1 GPa, 0.08 GPa, 0.06 GPa, 0.04 GPa, 0.02 GPa, 0.01 GPa, 0.008 GPa, 0.006 GPa, 0.004 GPa, 0.002 GPa, 0.001 GPa, 0.0008 GPa, 0.0006 GPa, 0.0004 GPa, 0.0002 GPa, or 0.0001 GPa. In some embodiments, the hydrogel has an elastic modulus of less than 100,000 pascals (Pa).

In certain embodiments, the particle has at least one cross-sectional dimension of between about 50 nm and about 100 μm (e.g., from 1 μm to 50 μm). For example, the complex is substantially spherical and has a diameter of between about 1 μm and 100 μm (e.g., from 2 μm to 80 μm, from 3 μm to 50 μm, from 4 μm to 25 μm, from 5 μm to 15 μm, from 8 μm to 12 μm, or about 10 μm). In some embodiments, the average diameter of a plurality of complexes is between about 1 μm and 100 μm (e.g., from 2 μm to 80 μm, from 3 μm to 50 μm, from 4 μm to 25 μm, from 5 μm to 15 μm, from 8 μm to 12 μm, or about 10 μm).

The binding moiety of the complex may be covalently or non-covalently attached to the hydrogel. In some embodiments, the binding moiety is attached through a linker, such as an avidin-biotin linker (e.g., a streptavidin-biotin linker). For example, the hydrogel is covalently conjugated with streptavidin, followed by non-covalent conjugation to a biotinylated binding moiety.

In certain embodiments, the polymer is a copolymer of alginate, e.g., with PEG or PMOXA. In such embodiments, the immune cell is, for example, an NK cell, CIK cell, TIL cell, HS cell (undifferentiated and differentiated), MS cell (undifferentiated and differentiated), iPS cell (undifferentiated and differentiated), or ES cell (undifferentiated and differentiated). In other such embodiments, the binding moiety is a cytokine, e.g., IL-1, IL-2, IL-3, IL-6, IL-7, IL-12, IL-15, IL-18, IL-21, TNF-α, or IFN-γ, or the binding moiety is chemokine (C-X-C motif) ligand 12 or low-density lipoprotein. In further such embodiments, the cell surface component is CD19, CD133, CD134, CD335, CD337, Delta-like ligand (e.g., DLL-Fc, DLL-1, or DLL-4), WNT3, stem cell factor, or thrombopoietin. For example, the binding moiety is anti-CD19, anti-CD133, anti-CD134, anti-CD335, anti-CD337, anti-Delta-like ligand (e.g., anti-DLL-Fc, anti-DLL-1, or anti-DLL-4), anti-WNT3, anti-stem cell factor, or anti-thrombopoietin.

In a further aspect, the invention features a method of generating a population of expanded immune cells by contacting a starting population of immune cells with a plurality of particles of the invention, wherein the contact is operative to induce a metabolic change in the starting population of immune cells, thereby generating a population of expanded immune cells.

In certain embodiments, the particles change from a solid matrix into a solution or suspension, e.g., in response to a sufficient decrease of cationic concentration in the environment of the polymer. In other embodiments, the particles are administered to a culture comprising the population of immune cells at a particle-to-cell ratio from 1:20 to 20:1. For example, the particle-to-cell ratio is a particle-to-immune cell ratio, e.g., about 5:1. Alternatively, the particle-to-cell ratio is a complex-to-peripheral blood mononuclear cell (PBMC) ratio, e.g., about 10:1. In certain embodiments, the population of expanded immune cells includes 100-fold the number of immune cells relative to the starting population. In other embodiments, the population of expanded immune cells includes activated immune cells.

In one aspect, the invention features a complex including a hydrogel and a binding moiety, wherein the hydrogel comprises an alginic acid-polyethylene glycol (PEG) copolymer; and the binding moiety is configured to bind a cell surface component of a T cell. The alginic acid-PEG copolymer can change from a solid matrix into a solution or suspension in response to a sufficient decrease of cationic concentration in the environment of the polymer. For example, the decrease in the cationic concentration in the environment of the polymer can be caused by the presence of EDTA, EGTA, sodium citrate, BAPTA, crown ether, cryptand, phenanthroline sulfonate, dipyridyl sulfonate, dioxane, DME, diglyme, or triglyme. In some embodiments, the cation is Li⁺, Mg²⁺, Ca²⁺, Sr²⁺, Ba²⁺, Zn²⁺, Cu²⁺, or Al³⁺.

In some embodiments, the complex completely liquefies in response to a sufficient decrease of cationic concentration in the environment of the complex, e.g., the complex does not further include a separation unit, such as a magnetic bead substrate or magnetic particles.

In some embodiments, the elastic modulus of the hydrogel is sufficient both to induce expansion and skew the phenotype of an expanding population. For example, the hydrogel may have an elastic modulus of less than 100,000 pascals (Pa). Such properties can be imparted by the molecular structure of the copolymer. For example, the alginic acid-PEG copolymer can include a multi-arm PEG molecule (e.g., a four-arm PEG molecule).

The geometry of the hydrogel complex influences its contact with cells and therefore may impact expansion. In some embodiments, the complex has at least one cross-sectional dimension of between about 50 nm and about 100 μm (e.g., from 1 μm to 50 μm). For example, the complex may be substantially spherical and have a diameter of between about 1 μm and 100 μm (e.g., from 2 μm to 80 μm, from 3 μm to 50 μm, from 4 μm to 25 μm, from 5 μm to 15 μm, from 8 μm to 12 μm, or about 10 μm). In some embodiments, the average diameter of a plurality of complexes is between about 1 μm and 100 μm (e.g., from 2 μm to 80 μm, from 3 μm to 50 μm, from 4 μm to 25 μm, from 5 μm to 15 μm, from 8 μm to 12 μm, or about 10 μm).

The binding moiety of the complex may be covalently or non-covalently attached to the hydrogel (e.g., the hydrogel particle, e.g., covalently attached to an alginic acid domain of the alginic acid-PEG copolymer). In some embodiments, the binding moiety can be can be attached through a linker, such as an avidin-biotin linker (e.g., a streptavidin-biotin linker). For example, the hydrogel can be covalently conjugated with streptavidin, followed by non-covalent conjugation to a biotinylated binding moiety.

In some embodiments, the surface of the hydrogel complex includes at least one binding moiety per square μm (e.g., at least 1 binding moiety per square μm, at least 2 binding moieties per square μm, at least 3 binding moieties per square μm, at least 4 binding moieties per square μm, at least 5 binding moieties per square μm, at least 10 binding moieties per square μm, at least 20 binding moieties per square μm, at least 30 binding moieties per square μm, at least 40 binding moieties per square μm, or at least 50 binding moieties per square μm). In some instances, one or more of the binding moieties is an antibody or antigen-binding fragment thereof.

The binding moiety may be a signal 1 stimulus (e.g., anti-CD3) or a signal 2 stimulus (e.g., anti-CD28). In complexes having both a signal 1 stimulus (e.g., anti-CD3) and a signal 2 stimulus (e.g., anti-CD28), the molar ratio of the signal 1 stimulus and the signal 2 stimulus can be between about 1:100 and about 100:1 (e.g., from 1:80 to 80:1, from 1:60 to 60:1, from 1:50 to 50:1, from 1:40 to 40:1, from 1:30 to 30:1, from 1:20 to 20:1, from 1:10 to 10:1, from 1:5 to 5:1, from 1:2 to 2:1, or about 1:1). In some embodiments, the signal 1 stimulus is antigen-specific.

Alternatively, a complex may include three or more types of binding moieties. For example, in some embodiments, the complex includes a signal 1 stimulus (e.g., anti-CD3), a signal 2 stimulus (e.g., anti-CD28), and an additional stimulus, such as an activating stimulus, a suppressive stimulus, or a polarizing stimulus, such as a cytokine (e.g., a surface-bound cytokine, e.g., a trans-presented interleukin).

A binding moiety can be an antibody or antigen-binding fragment thereof. For example, a binding moiety can be a monoclonal antibody or antigen-binding fragment thereof, a Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′) 2) molecule, a domain antibody, a diabody, a triabody, an affibody, a domain antibody, a SMIP, a nanobody, a Fv fragment, a Fab fragment, a F(ab′) 2 molecule, or a tandem scFv (taFv) fragment. In some embodiments, the antibody or antigen-binding fragment thereof is anti-CD2, anti-CD3, anti-CD27, anti-CD28, anti-CD46, or anti-CD137.

In one aspect, the invention features a complex including a hydrogel particle and at least two binding moieties, wherein the hydrogel particle includes an alginic acid-PEG copolymer and Ca²⁺, and the binding moieties include anti-CD3 and anti-CD28, wherein the alginic acid-PEG copolymer changes from a solid matrix into a solution or suspension in response to a sufficient decrease of Ca²⁺ concentration in the environment of the copolymer.

In some embodiments, the complex completely liquefies in response to a sufficient decrease of cationic concentration in the environment of the complex, e.g., the complex does not further include a separation unit, such as a magnetic bead substrate or magnetic particles.

In another aspect, the invention features a complex of the invention produced by atomization of an alginic acid-PEG copolymer. For example, an alginic acid-PEG copolymer solution can be passed through an atomizer to produce an atomized spray. The spray can be directed into a receiving solution having a cationic concentration sufficient to result in crosslinking of the alginic acid-PEG copolymer, thereby generating an alginic acid-PEG particle (e.g., a microparticle or nanoparticle). To produce the complex, the particle is then conjugated with a binding moiety.

In some embodiments, the alginic acid-PEG copolymer solution flows through the atomizer at a volumetric percentage from 30% to 90%. The droplets may be produced in the atomizer by an injection of gas (e.g., pressurized gas, e.g., pressurized air or nitrogen), e.g., from 1 to 200 pounds per square inch (psi). In some embodiments, the alginic acid-PEG copolymer solution may flow through the atomizer at a rate from 0.1 to 100 mL per minute. In other embodiments, the rate of gas flow through the atomizer is independent from the rate of alginic acid-PEG copolymer solution, such as in the case of an external mix atomizer. In further embodiments, the atomizer produces a round spray pattern, e.g., at a spray angle from 10° to 30°.

In some embodiments, the complex produced by atomization completely liquefies in response to a sufficient decrease of cationic concentration in the environment of the complex, e.g., the complex does not further include a separation unit, such as a magnetic bead substrate or magnetic particles.

In another aspect, the invention provides a method of producing an alginic acid-PEG particle by passing an alginic acid-PEG copolymer solution through an atomizer to produce an atomized solution. The atomized solution is contacted with a receiving solution having a cation, which generates an alginic acid particle. In some embodiments, a binding moiety is further conjugated to the alginic acid-PEG particle to produce a hydrogel complex. The hydrogel complex can have an elastic modulus of less than 100,000 Pa. In some embodiments, the binding moiety binds to a cell surface component of a T cell.

In some embodiments, the alginic acid-PEG copolymer solution flows through the atomizer at a volumetric percentage from 30% to 90%. The droplets may be produced in the atomizer by an injection of gas (e.g., pressurized gas, e.g., pressurized air or nitrogen), e.g., from 1 to 200 pounds per square inch (psi). In some embodiments, the alginic acid-PEG copolymer solution may flow through the atomizer at a rate from 0.1 to 100 mL per minute. In other embodiments, the rate of gas flow through the atomizer is independent from the rate of alginic acid-PEG copolymer solution, such as in the case of an external mix atomizer. In further embodiments, the atomizer produces a round spray pattern, e.g., at a spray angle from 10° to 30°.

In another aspect, the invention features a method of generating a population of expanded T cells, wherein the method involves contacting a starting population of T cells with a plurality of complexes of any of the preceding aspects, wherein the contact induces a metabolic change in the starting population of T cells, thereby generating a population of expanded T cells. In some embodiments, the method further includes liquefying some of all of the complexes by exposing a cation chelator to the complexes and the population of expanded T cells. In some embodiments, the complex completely liquefies in response to a sufficient decrease of cationic concentration in the environment of the complex, e.g., the complex does not further include a separation unit, such as a magnetic bead substrate or magnetic particles.

The complexes can be administered to a culture having the population of T cells at a complex-to-cell ratio from 1:1 to 20:1, or from 1:20 to 20:1 (e.g., a ratio of complexes to any phenotype of cells, including T cells and other cell types, for example, B cells, macrophages, dendritic cells, neutrophils, or stromal cells). Additionally or alternatively, the complexes can be administered to a culture comprising the population of T cells at a complex-to-T cell ratio from about 1:1 to 20:1 (e.g., about 5:1). Additionally or alternatively, the complexes can be administered to a culture comprising the population of T cells at a complex-to-peripheral blood mononuclear cell (PBMC) ratio from about 1:1 to about 20:1 (e.g., about 10:1).

Methods of the invention enable expansion of a T cell population such that the expanded T cells differ in phenotype compared to the starting population. For example, an expanded population may have a greater number of activated T cells compared to the starting population. Additionally or alternatively an expanded population may include a greater number or percentage of CD8⁺ T cells than the starting population. Conversely, the population of expanded T cells may include a lower number or percentage of CD4⁺ T cells than the starting population. Additionally or alternatively, the population of expanded T cells may include a greater CD8-to-CD4 T cell ratio than the starting population. In general, the population of expanded T cells will include a greater overall number of T cells relative to the starting population (e.g., 2-fold, 5-fold, 10-fold, 20-fold, 50-fold, or over 100-fold the number of T cells). All or a portion of the expanded T cells may have an activated phenotype. In some embodiments, the complex is completely liquefied in response to a sufficient decrease of cationic concentration in the environment of the complex.

In some embodiments, the resulting population of cells has at least 2% naïve T cells (e.g., at least 3%, at least 4%, at least 5%, at least 6%, at least 7%, at least 8%, at least 9%, at least 10%, or more, e.g., about 5%, about 10%, about 15%, or more). In some embodiments, the resulting population of cells has a greater number or percentage of naïve T cells relative to a reference population (e.g., cells expanded by control beads, e.g., 10% more, 20% more, 50% more, 100% more, or greater, e.g., 2-fold more, 3-fold more, 4-fold more, 5-fold more, 10-fold more, or greater). Additionally or alternatively, the resulting population of cells may have a greater number or percentage of central memory T cells than the reference population (e.g., 10% more, 20% more, 30%, more, 40% more, 50% more, 75% more, 100% more, 150% more, 200% more, 300% more, 400% more, 500% more, 1,000% more, 5,000% more, 10,000% more, or greater). In some embodiments, the naïve T cells are CD45RA⁺ cells, CD45RA⁺CD62L⁺ cells, or CD45RA⁺CCR7⁺ cells. In other embodiments, the naïve T cells secrete lower quantities of IL-4 and/or IFN-γ than a reference population of cells (e.g., wherein the reference population is the starting population, a central memory cell population, an effector memory cell population, or an activated population).

In one embodiment, the population of T cells is isolated from a subject. In a different embodiment, the population of T cells is derived from a cell line. In one aspect, the starting population of T cells comprises a genetic modification, such as that resulting from a chimeric antigen receptor (CAR) modification.

In one aspect, the metabolic change induced in the T cells by contacting them with the complexes includes a biochemical or a morphological change. This change may be a greater frequency of cell division, a change in cytokine secretion profile (e.g., of IL-4 and/or IFN-γ), an increase in median cell diameter, a change surface molecule expression profile, or a change in cellular motility.

As used herein, the term “average” refers broadly to any representative value of a set of values or characteristic of a population of discrete objects. For example, an average diameter of a particle (e.g., nanoparticle or microparticle) or complex may refer to a mean, median, mode, or any weighted variant thereof, including average values derived from excluding outlying data. Unless otherwise specified, the “size” of a particle or complex is its diameter.

As used herein, the term “droplet” refers to a liquid product of an atomizer, whereas a “particle” refers herein to a solid (e.g., gel or hydrogel) spherical or substantially spherical construct (e.g., a nanoparticle or microparticle). A droplet becomes a particle upon solidification (e.g., gelling, e.g., upon exposure to a cation, resulting in alginate crosslinking).

As used herein, the term “complex” refers to a hydrogel construct (e.g., a particle, disk, rod, or other shape) that is associated with (e.g., conjugated to) one or more binding moieties.

As used herein, “volumetric percentage of liquid passed through an atomizer,” and grammatical variants thereof, refer to the volume of liquid passing through an atomizer relative to the total volumetric flow through the atomizer, including a gas. For example, the volumetric percentage Vₗ of liquid flowing through an atomizer over a given period of time is given as:

$${V}_{l} = \frac{1}{l + g} \times 100$$

where l is the volume of liquid passed through the atomizer over the given period of time, and g is the volume of gas passed through the atomizer over the given period of time.

As used herein, a “reference population” refers to any suitable control population of cells. For example, a characteristic of a population of cells can be compared to the starting population from which it has expanded. Alternatively, the reference population can be an untreated control or a control that has been treated (e.g., expanded) by an alternative means. Alternative means of T cell expansion include any conventional methods, such as use of soluble, plate-bound, and/or bead- or particle-bound antibodies or cytokines (e.g., T cell activating antibodies, such as anti-CD3 and/or anti-CD28). For example, a reference population of an expanded population of T cells can be generated using custom-made or commercially available control beads (e.g., DYNABEADS®).

### BRIEF DESCRIPTION OF THE DRAWINGS

FIG. 1: A schematic drawing showing a cross-sectional view of an exemplary spray apparatus.

FIGS. 2A-2D: Photomicrographs showing hydrogel particles of the invention. FIGS. 3A and 3B show the hydrogel particles prior to binding moiety conjugation at high density (FIG. 2A; 1.16×10⁸ particles/mL) and at low density (FIG. 2B; 1.16×10⁷ particles/mL). FIGS. 2C and 2D show the hydrogel particles after conjugation with anti-CD3 and anti-CD28 antibodies at a concentration of 4×10⁷ particles/mL. The scale bar in each image represents 10 μm.

FIG. 3: Graph showing T cell expansion by anti-CD3/anti-CD28 antibody-coated hydrogel complexes in comparison to control beads and untreated cells. Cells were treated with hydrogel complexes at complex-to-cell ratios of 10:1 and 5:1.

FIGS. 4A-4B: Bar graphs showing change in CD4 and CD8 expression in T cells after 9 days of expansion. FIG. 4A shows the change in the percentage of CD4⁺ cells as a result of hydrogel complex treatment versus control complex treatment. FIG. 4B shows the change in the percentage of CD4⁺ cells as a result of hydrogel complex treatment versus control complex treatment. Cells were treated with hydrogel complexes at complex-to-cell ratios of 10:1 and 5:1.

FIGS. 5A-5D: Graphs showing expression level activation markers in CD8⁺ T cells and CD4⁺ T cells over 9 days of expansion by hydrogel complexes relative to control beads. FIGS. 5A and 5B show the percentage of CD8⁺ T cells (FIG. 5A) and CD4⁺ T cells (FIG. 5B) that express CD25 over time. FIGS. 5C and 5D show the percentage of CD8⁺ T cells (FIG. 5C) and CD4⁺ T cells (FIG. 5D) that express CD69 over time.

FIGS. 6A-6D: Flow cytometry graphs representing data used to determine the level activation marker expression (CD25 and CD69) on CD8⁺ T cells (FIGS. 6A and 6C) and CD4⁺ T cells (FIGS. 6B and 6D). FIG. 6A shows CD25 expression by CD8⁺ T cells treated with control beads (top row) and hydrogel complexes at a 10:1 complex-to-cell ratio (bottom row) at day 2 of the expansion period (left column) and at day 5 of the expansion period (right column). FIG. 6B shows CD25 expression by CD4⁺ T cells treated with control beads (top row) and hydrogel complexes at a 10:1 complex-to-cell ratio (bottom row) at day 2 of the expansion period (left column) and at day 5 of the expansion period (right column). FIG. 6C shows CD69 expression by CD8⁺ T cells treated with control beads (top row) and hydrogel complexes at a 10:1 complex-to-cell ratio (bottom row) at day 2 of the expansion period (left column) and at day 5 of the expansion period (right column). FIG. 6D shows CD69 expression by CD4⁺ T cells treated with control beads (top row) and hydrogel complexes at a 10:1 complex-to-cell ratio (bottom row) at day 2 of the expansion period (left column) and at day 5 of the expansion period (right column). The population of each graph is from a parent gate on either CD4⁺ or CD8⁺ events after gating on single cells. Side scatter (SSC) is plotted versus activation marker (CD25 or CD69).

FIG. 7: A graph showing modulation of ligand and ligand density modulates T cell expansion.

FIG. 8: A series of graphs showing modulation of ligand and ligand density modulates T cell phenotype.

FIG. 9: A series of graphs showing modulation of ligand and ligand density modulates memory phenotypes.

### DETAILED DESCRIPTION OF THE INVENTION

The invention provides novel particles and hydrogel complexes for modulation of immune cells. The particles and complexes may be employed with a separation unit, such as a magnetic bead, inside them. Residual magnetic particles represent a possible toxicological risk. Additionally, removal of residual magnetic particles requires addition of magnetic separation steps, which increase workflow costs, time, and complexity, and lead to cell loss, reducing the total number of recovered cells following expansion. These workflow challenges are significant for cell therapy bioprocessing applications, hence the impetus for developing a paramagnetic bead-free hydrogel particle design. For example, existing T cell expansion methods utilize magnetic particles (e.g., paramagnetic particles) to mediate cell separation. For T cell expansion methods that do not require cell separation, the paramagnetic particles also introduce workflow complexity and challenges.

This invention provides a particle or hydrogel complex that does not require a substrate such as magnetic particles and that binds to and modulates an immune cell, e.g., expands a desired T cell population. In certain embodiments, the particle or complex can be gently dissociated after expansion, e.g., resulting in a pure population of expanded T cells.

Also provided herein are methods for synthesizing particles or hydrogel complexes of the invention, for example, by spraying an atomized droplet suspension of a cross-linkable copolymer into a receiving solution having cations to crosslink the copolymer to form a hydrogel particle that can then be bound to a binding moiety to form a complex. Methods of using such particles or complexes as part of adoptive T cell therapy methods and systems are also provided by the present invention.

### Binding Moieties

The invention features a complex including a binding moiety attached to a hydrogel structure (e.g., a hydrogel particle). In general, binding moieties are located on the surface of the hydrogel structure.

A binding moiety may bind to another binding moiety, e.g., an antibody or antigen-binding fragment thereof, bound to the target cells. For example, the binding moiety may be avidin or streptavidin, and it may bind to a target cell that has been labeled with biotin, e.g., via biotinylated antibodies. Other such binding moieties include protein A, protein G, and anti-species antibodies (e.g., goat anti-rabbit antibodies) that bind to antibodies bound to a target cell.

### Binding Moieties

Binding moieties can bind to a surface component of an immune cell, e.g., a T cell. Exemplary surface components are CD2, CD3, CD19, CD24, CD27, CD28, CD31, CD34, CD45, CD46, CD80, CD86, CD133, CD134, CD135, CD137, CD160, CD335, CD337, CD40L, ICOS, GITR, HVEM, Galtectin 9, TIM-1, LFA-1, PD-L1, PD-L2, B7-H3, B7-H4, ILT3, ILT4, CDTL-4, PD-1, BTLA, MHC-I, MHC-II, Delta-like ligand (e.g., DLL-Fc, DLL-1, or DLL-4), WNT3, stem cell factor, and thrombopoietin. The binding moiety may be an antibody or antigen binding fragment thereof or another molecule that binds to the surface component, e.g., a cytokine. Suitable cytokines include, for example, IL-1, IL-2, IL-3, IL-6, IL-7, IL-12, IL-15, IL-18, IL-21, TNF-α, and IFN-γ. Exemplary antibodies or antigen binding fragments thereof include anti-CD2, anti-CD3, anti-CD19, anti-CD24, anti-CD27, anti-CD28, anti-CD31, anti-CD34, anti-CD45, anti-CD46, anti-CD80, anti-CD86, anti-CD133, anti-CD134, anti-CD135, anti-CD137, anti-CD160, anti-CD335, anti-CD337, anti-CD40L, anti-ICOS, anti-GITR, anti-HVEM, anti-Galtectin 9, anti-TIM-1, anti-LFA-1, anti-PD-L1, anti-PD-L2, anti-B7-H3, anti-B7-H4, anti-ILT3, anti-ILT4, anti-CDTL-4, anti-PD-1, anti-BTLA, anti-MHC-I, anti-MHC-II, anti-Delta-like ligand (e.g., anti-DLL-Fc, anti-DLL-1, or anti-DLL-4), anti-WNT3, anti-stem cell factor, or anti-thrombopoietin. The binding moiety may be a chemokine (C-X-C motif) ligand 12 or low-density lipoprotein. Preferably, this binding event can lead to signal transduction within the immune cells, e.g., T cell, e.g., resulting in modulation, such as activation, expansion (i.e., proliferation), and/or other phenotypic change of the target cell (e.g., polarization (e.g., polarization toward a Th1, Th2, Th17, Treg, or another T cell sub-phenotype) or expansion in the absence of activation, e.g., retention of a naïve phenotype (e.g., CD45RA⁺)). Several immune, e.g., T cell, surface molecules are known to have such downstream effects. For T cells, ligands that induce clustering of the T cell receptor (signal 1) can stimulate a T cell to proliferate and, depending on the presence, concentration, affinity, or avidity of a secondary signal (signal 2), the T cell may differentiate or polarize towards a particular phenotype. The signal 1 agent of the present invention can be anti-CD3, and the signal 2 agent of the present invention can be anti-CD28. Other examples of signal 1 stimuli include MHC-I or MHC-II, as well as agonists of various other T cell receptor components known in the art. Other examples of signal 2 stimuli include antigen-presenting cell surface molecules that are agonists of co-stimulatory molecules (e.g., CD80, CD86, CD40, ICOSL, CD70, OX40L, 4-1 BBL, GITRL, LIGHT, TIM3, TIM4, ICAM1, or LFA3), antibodies toward T cell costimulatory surface molecules other than CD28 (e.g., CD40L, ICOS, CD27, OX40, 4-1BB, GITR, HVEM, Galectin 9, TIM-1, LFA-1, and CD2), antigen-presenting cell surface molecules that are agonists of co-inhibitory molecules (e.g., CD80, CD86, PD-L1, PD-L2, B7-H3, B7-H4, HVEM, ILT3, or ILT4), and antibodies against co-inhibitory molecules (e.g., CDTL-4, PD-1, BTLA, or CD160). Signal 2 stimulation has been shown to affect multiple aspects of T cell activation. In general, signal 2 stimulation is thought to lower the concentration of anti-CD3 required to induce a proliferative response in cultures and enhances cytokine production to direct T cell differentiation pathways. Importantly, costimulation can help to activate cytolytic potential of CD8⁺ T cells. Other molecules, including but not limited to CD2 and CD137, can be targeted to activate and expand various T cell populations, such naïve and memory T cells, T helper cells, regulatory T cells, Natural Killer T cells, and Cytotoxic T Lymphocytes from mouse and human samples. Additional examples of antibodies, ligands, and other agents useful as signal 1 and signal 2 stimuli for use in the present invention are described in WO 2003/024989.

The particles or complexes of the invention may include two or more different binding moieties. For example, the binding moieties may bind to at least one activation receptor and a cognate ligand, which in turn can work in tandem with a co-stimulatory signal and/or cytokine to elicit growth and activation of an immune cell. The following discussion relates to T cells, but similar processes are known for other immune cells.

### Antigen-Specific T Cell Engagement

In one aspect of the invention, a binding moiety may bind a T cell receptor in an antigen-specific manner analogous to the binding that occurs between the T cell receptor and a peptide-MHC on an antigen presenting cell. Synthetic methods to engage a T cell in an antigen specific manner (i.e., in the absence of a natural antigen presenting cell) include MHC class I and MHC class II multimers (e.g., dimers, tetramers, and dextramers). Illustrative examples are described in U.S. Pat. No. 7,202,349 and U.S. 2009/0061478. Multimerization of MHC-peptide complexes functions to enhance avidity of interaction between peptide-MHC and T cells, which increases the potency of signal 1 transduction. Another means to achieve multivalent presentation of antigen-specific T cell receptor ligands is by tethering the ligands to a surface of a hydrogel structure (e.g., a hydrogel particle). Affinity, in contrast to avidity, describes the strength of binding of each individual molecule. The affinity of peptide-MHC for a T cell receptor can vary dramatically and dictates the downstream effect of signal 1 stimuli, as discussed below. Antigens, and peptides thereof, for use in the present invention include, but are not limited to melanoma antigen recognized by T cells (MART-1), melanoma GP100, the breast cancer antigen, Her-2/Neu, and mucin antigens. Other relevant antigens and sources thereof are described, for example, in U.S. Pat. No. 8,637,307.

In some embodiments, there are initial activation signaling events determining downstream outcomes, i.e., activation of multiple immune cell types is modulated by the ligand, half-life of receptor-ligand interactions, and ligand concentrations. The following discussion relates to T cells, but similar processes are known for other immune cells.

### Effects of the Degree of T Cell Binding

The relative degree of signal 1 and signal 2 binding can influence TCR signal transduction and lead to varying downstream phenotypic effects. For example, high affinity with low avidity signal 1, in the absence of signal 2, primes a naïve CD4⁺ T cell to turn on FoxP3 expression and differentiate toward a regulatory phenotype (Gottschalk et al., Journal of Experimental Medicine 207 (2010): 1701). Alternatively, in response to a high avidity signal 1 stimulus without sufficient signal 2 stimulus, a naïve T cell may be more likely to undergo exhaustion, leading to functional anergy (see Ferris et al., J Immunol August 15; 193 (2014): 1525-1530). Either of these effects would be undesirable in the context of cancer adoptive immunotherapy but may be helpful in when priming regulatory T cells for autoimmune treatment. Thus, the relative contribution of signal 1 and signal 2 can be rationally modulated, depending on the application, by exposing the functionalized complex to a desired ratio of binding moieties.

The binding moieties may be coupled to the same surface or to separate surfaces. In a preferred embodiment, a signal 1 stimulus and a signal 2 stimulus are immobilized on a surface (e.g., a surface of a hydrogel particle) at a 1:1 ratio. In certain aspects of the present invention, a signal 1 stimulus and a signal 2 stimulus are immobilized on the surface at a ratio of other than 1:1 (e.g., between about 1:100 and about 100:1, between about 1:10 and 10:1, between about 1:2 and 2:1). Alternatively, the ratio of signal 1 stimulus to signal 2 stimulus immobilized on the surface is greater than 1:1, or less than 1:1.

The effect of relative strength of signaling between signal 1 and signal 2 also depends on the activation state of the target T cell. For instance, naïve T cells react differently to T cell receptor stimulation and costimulation than antigen-experienced T cells. A skilled artisan would appreciate this effect when selecting a configuration of binding moieties of the present invention, especially in cases of chronic infection or aberrant immune tolerance, and configure the binding moiety accordingly.

### Hydrogels

Particle and complexes of invention include a hydrogel. In some embodiments, the hydrogel can be dissolved (i.e., liquefied) by changing the ionic composition of their environment. Hydrogels of the invention can be formed from natural polymers, synthetic polymers, and copolymers thereof. Exemplary natural polymers are alginate, agarose, carrageenan, chitosan, dextran, carboxymethylcellulose, heparin, hyaluronic acid, polyamino acids, collagen, gelatin, fibrin, fibrous protein-based biopolymers (e.g., silk, keratin, elastin, and resilin), and any combination thereof. Synthetic polymers include poly(ethylene glycol) (PEG), poly(2-methyl-2-oxazoline) (PMOXA), poly(ethylene oxide) (PEO), poly(vinyl alcohol) (PVA), and poly(acrylamide) (PAAm), poly(n-butyl acrylate), poly-(α-esters), poly(glycolic acid) (PGA), poly(lactic-co-glycolic acid) (PLGA), poly(L-lactic acid) (PLLA), poly(N-isopropylacrylamide) (pNIPAAM), butyryl-trihexyl-citrate, di(2-ehtylhexyl)phthalate, di-iso-nonyl-1,2-cyclohexanedicarboxylate, expanded polytetrafluoroethylene (PTFE), ethylene vinyl alcohol copolymer, poly(hexamethylene diisocyanate), high density poly(ethylene) (PE), highly crosslinked PE, poly(isophorone diisocyanate), low density PE, poly(amide), poly(acrylonitrile), poly(carbonate), poly(caprolactone diol), poly(D-lactic acid), poly(dimethylsiloxane), poly(dioxanone), poly(ethylene), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), ultrahigh molecular weight PE, and any combination thereof. Specific copolymers include an alginate-PEG copolymer or an alginate-PMOXA copolymer.

In one embodiments, hydrogels of the invention can be formed from alginic acid (i.e., alginate) conjugated to a polyalkylene oxide, e.g., polyethylene glycol (PEG), as generally described in WO 2012/106658. PEG can be multifunctional PEG (e.g., multi-armed PEG, e.g., 4-arm PEG). Conjugation of alginic acid to multifunctional PEG (e.g., 4-arm PEG) confers greater mechanical strength compared with that achieved solely by ionic crosslinking of alginate. Thus, the mechanical properties (e.g. stiffness) can be modulated by increasing the number of functional groups on each PEG molecule. PEG is useful as part of the present invention because of its superior hydrophilic properties, which prevent protein adsorption to the complex of the present invention. Adsorption of serum proteins onto the complex can result in aberrant signaling pathways in adjacent cells, such as those caused by Fc receptor engagement. The hydrophilic properties of PEG are also functional to maintain a high diffusivity within the complex interior, such that ionic chelators can rapidly access the complex interior to sequester rigidity-maintaining cations quickly. Thus, incorporation of branched PEG molecules within the hydrogel ensures a rapid dissolution of the hydrogel structure upon exposure to appropriate stimuli. Similar effects may be achieved by forming a copolymer of PEG with a polymer other than alginate, as described herein. Alternatively, any other biocompatible hydrophilic polymer (e.g. polyvinyl pyrrolidone, polyvinyl alcohol, and copolymers thereof) can be substituted for PEG (see, e.g., U.S. Pat. No. 7,214,245), e.g., with alginate or another polymer. PMOXA may also be included in a copolymer with alginate or another polymer as described herein.

A hydrogel complex of the invention can have one or more mechanical properties (e.g., elastic modulus, Young's modulus, compression modulus, or stiffness) suitable for immune cell modulation, e.g., T cell expansion. For example, the mechanical properties of the hydrogel can be tailored to be suitable to allow a population of T cells (e.g., a population containing expanded T cells) to retain one or more characteristics of a naïve phenotype (e.g., as described in the Methods section) after contacting a complex of the invention. An elastic modulus of a hydrogel, e.g., that is suitable to allow a population of T cells to retain one or more characteristics of a naïve phenotype, may be between 100 pascals (Pa) and 100,000,000 Pa (e.g., from 100 Pa to 1,000 Pa, from 1,000 Pa to 10,000 Pa, between 10,000 Pa to 100,000 Pa, between 100,000 Pa and 1,000,000 Pa, 1,000,000 Pa and 10,000,000 Pa, or 10,000,000 Pa and 100,000,000 Pa, e.g., less than 1,000,000 Pa, less than 900,000 Pa, less than 800,000 Pa, less than 700,000 Pa, less than 600,000 Pa, less than 500,000 Pa, less than 400,000 Pa, less than 300,000 Pa, less than 200,000 Pa, less than 100,000 Pa, less than 50,000 Pa, or less than 10,000 Pa). In other embodiments, the elastic modulus of the particle or complex is less than 1 gigapascal (GPa), e.g., 0.8 GPa, 0.6 GPa, 0.4 GPa, 0.2 GPa, 0.1 GPa, 0.08 GPa, 0.06 GPa, 0.04 GPa, 0.02 GPa, 0.01 GPa, 0.008 GPa, 0.006 GPa, 0.004 GPa, 0.002 GPa, 0.001 GPa, 0.0008 GPa, 0.0006 GPa, 0.0004 GPa, 0.0002 GPa, or 0.0001 GPa.

In other embodiments, a mechanical property of the hydrogel can be configured to expand CD8⁺ T cells preferentially (e.g., relative to CD4⁺ T cells, or relative to a total cell population, e.g., total CD3+ T cells or total lymphocytes). For example, an elastic modulus of a hydrogel configured to expand CD8⁺ T cells preferentially is between 100 pascals (Pa) and 100,000,000 Pa (e.g., from 100 Pa to 1,000 Pa, from 1,000 Pa to 10,000 Pa, between 10,000 Pa to 100,000 Pa, between 100,000 Pa and 1,000,000 Pa, 1,000,000 Pa and 10,000,000 Pa, or 10,000,000 Pa and 100,000,000 Pa, e.g., less than 1,000,000 Pa, less than 900,000 Pa, less than 800,000 Pa, less than 700,000 Pa, less than 600,000 Pa, less than 500,000 Pa, less than 400,000 Pa, less than 300,000 Pa, less than 200,000 Pa, less than 100,000 Pa, less than 50,000 Pa, or less than 10,000 Pa).

A reference to alginic acid is also a reference to a salt form, e.g., sodium alginate, unless otherwise noted. The alginic acid content of the polymeric moiety will influence the stiffness of the polymeric moiety according to known principles (e.g., cation content of the polymeric moiety). The stiffness of alginate polymeric moieties can be varied while maintaining a constant or near-constant density according to known methods.

Polyalkylene oxides, e.g., PEG and polypropylene oxide, are known in the art. Linear or branched, e.g., 4-arm or 8-arm, polyalkylene oxides, e.g., PEG, may be employed. The polyalkylene oxide, e.g., PEG, preferably has a molecular weight between 10 kDa and 20 kDa. An exemplary ratio of polyalkylene oxide, e.g., PEG, to alginic acid is 1:2 by weight.

Alginic acid naturally possesses multiple carboxyl groups that provide convenient groups for conjugation to polyalkylene oxide, e.g., PEG, and/or binding moieties. The polyalkylene oxide, e.g., PEG, and binding moiety will naturally possess or be modified to possess an appropriate group to conjugate to a carboxyl group. Suitable groups include amine groups, which are often found in binding moieties that include amino acids or can be introduced into binding moieties and polyalkylene oxides, e.g., PEG. For example, amine-terminated polyalkylene oxide, e.g., PEG, can be employed. In other embodiments, a linker may be used to conjugate appropriate groups on the polyalkylene oxide, e.g., PEG, or binding moiety to carboxyl groups on the alginic acid. In the hydrogel, a single polyalkylene oxide, e.g., PEG, may be conjugated to one or more alginic acid molecules. When a polyalkylene oxide binds to more than one alginic acid, the number of such crosslinks in the composition may or may not be sufficient to form a gel. The binding moiety can bind to either the alginic acid directly or to a polyalkylene oxide, e.g., PEG, bound to alginic acid.

The hydrogel forms by noncovalent crosslinking of the alginic acid with a cation, e.g., Li⁺, Mg²⁺, Ca²⁺, Sr²⁺, Ba²⁺, Zn²⁺, Cu²⁺, or Al³⁺. A preferred cation is Ca²⁺. Gelation of hydrogels of the invention may be reversed by contact with a chelator for the cation, e.g., EDTA, EGTA, sodium citrate, BAPTA, crown ether, cryptand, phenanthroline sulfonate, dipyridyl sulfonate, dioxane, DME, diglyme, or triglyme. Preferably, the chelator is a bioinert molecule such as EDTA, which is well-known not to interfere with cell growth and proliferation pathways at concentrations relevant for complex dissolution.

For polymers other than alginate and/or PEG, methods are known in the art to bind binding moieties and to form copolymers in manner analogous to those used for alginate and PEG.

### Size and Shape of the Complex

The complex can be any shape compatible with contacting the surface of an immune cell, e.g., T cell. For this reason, it is often preferable for a complex to have a high surface area to volume ratio to maximize the available binding surface. Artificial antigen presenting cell platforms having different sizes and shapes have been evaluated for various functional benefits (see Fadel et al., Nano Letters 8 (2008): 2070-2076; Sunshine et al., Biomaterials 35 (2014): 269-277). The shape of the structure may be any suitable shape, such as elongated like a wire, tubular, i.e., having a lumen, planar, or spherical. In some embodiments, the complex can have a surface configured to replicate the immune synapse between an antigen presenting cell and a T cell. Immune synapses can range in size from less than 50 nm to about 20 μm. In some embodiments, the complex of the present invention is a particle, e.g., that is spherical. In most embodiments, the diameter is less than 1,000 μm. For example, the diameter of the complex can be between 50 nm and 20 μm (e.g., between 100 nm and 15 μm, between 200 nm and 14 μm, between 500 nm and 13 μm, between 1 μm and 12 μm, or about 10 μm). For example, the complex can be the size of antigen presenting cells, such as dendritic cells or macrophage, which range from about 10-20 μm. Alternatively, the complex can include a larger matrix, such as a porous scaffold, which can be mechanically exposed, e.g. dipped, into a suspension of cells. Methods for synthesizing such scaffolds, including by using alginate, are known in the art.

### Synthesis

Compositions of the invention can be synthesized by any suitable means. Methods of the invention include synthesizing a copolymer (e.g., alginic acid-PEG) to form a copolymer solution.

Alginic acid, or another polymer, can be present in the copolymer solution in a percentage (e.g., a weight-by-volume percentage) between 0.01 and 10% (e.g., from 0.1% to 0.15%, from 0.15% to 0.2%, from 0.2% to 0.3%, from 0.3% to 0.4%, from 0.4% to 0.5%, from 0.5% to 0.6%, from 0.6% to 0.7%, from 0.7% to 0.8%, from 0.8% to 0.9%, from 0.9% to 1.0%, from 1.0% to 2%, from 2% to 2.5%, from 2.5% to 3%, from 3% to 4%, from 4% to 5%, from 5% to 7.5%, or from 7.5% to 10%, e.g., about 0.2%, 0.25%, 0.3%, 0.35%, 0.4%, 0.45%, 0.5%, 0.6%, 0.7%, 0.75%, 0.8%, 0.9%, 1.0%, 1.5%, 2.0%, 2.25%, 2.5%, 3%, 3.5%, 4%, 4.5%, 5%, or 10%).

Suitable alginic acid is a medium viscosity alginic acid (e.g., a 1-100 kDa medium viscosity alginic acid, e.g., 20 kDa medium viscosity alginic acid). Medium viscosity alginic acid (e.g., 20 kDa medium viscosity alginic acid) may have a viscosity in water or aqueous solution from 1 to 100,000 centipoise (cP; e.g., from 1 to 50 cP, from 50 to 100 cP, from 100 to 200 cP, from 200 to 500 cP, from 500 to 1,000 cP, from 1,000 to 5,000 cP, from 5,000 to 10,000 cP, from 10,000 to 20,000 cP, from 20,000 to 30,000 cP, from 30,000 to 40,000 cP, from 40,000 to 50,000 cP, or from 50,000 to 100,000 cP), depending on its concentration and/or composition (e.g., conjugation as a copolymer, e.g., as an alginic acid-PEG copolymer).

Methods of synthesizing a copolymer by conjugating a monomer (e.g., alginic acid to an alkylene oxide, e.g., PEG, e.g., multi-arm PEG, e.g., 4-arm PEG) are known in the art. For example, alginate-PEG copolymer can be synthesized using aminated PEG combined in a batch reaction of alginic acid, 1-ethyl-3-(-3-dimethylaminopropyl) carbodiimide hydrochloride (EDC), and sulfo-N-hydroxysuccinimide (NHS), which conjugates PEG to carboxylate groups on alginic acid. In some embodiments, PEG (e.g. 4-arm PEG-amine) is conjugated to alginic acid in a molar ratio of 1:4, 1:3, 1:2, 1:1, 2:1, 3:1, 4:1, or greater. In some embodiments, PEG (e.g. 4-arm PEG-amine) is conjugated to alginic acid at a mass ratio of about 2:1. For example, in some embodiments, 10-100 mg/mL alginic acid (e.g., about 22.5 mg/mL alginic acid) is reacted with 5-50 mg/mL PEG-amine (e.g., 4-arm PEG-amine; e.g., about 11.25 mg/ml PEG-amine, e.g., 4-arm PEG-amine). Conditions for suitable conjugation reactions are known in the art. In some embodiments, alginic acid is conjugated to PEG at room temperature in a solution containing 0.5 mg/ml to 2.0 mg/ml sulfo-NHS (e.g., 1.1 mg/ml sulfo-NHS) and 0.1 mg/ml to 1.0 mg/ml EDC (e.g., about 0.4 mg/ml EDC) for 12-24 hours (e.g., 18 hours, e.g., overnight).

The viscosity of the resulting copolymer solution will range depending on the copolymer concentration. For example, a copolymer solution having an alginic acid-PEG concentration from 30 to 40 mg/mL may have a viscosity from 50 to 50,000 cP (e.g., from 1 to 50 cP, from 50 to 100 cP, from 100 to 200 cP, from 200 to 500 cP, from 500 to 1,000 cP, from 1,000 to 5,000 cP, from 5,000 to 10,000 cP, from 10,000 to 20,000 cP, from 20,000 to 30,000 cP, from 30,000 to 40,000 cP, from 40,000 to 50,000 cP)

The present invention provides methods of synthesizing hydrogel complexes using a spray apparatus. In some embodiments, the copolymer is sprayed into a receiving solution that is a cationic solution (e.g., a solution having a Ca²⁺ concentration from 0.1 M to 100 M, e.g., from 0.5 M to 10 M, from 1 M to 5 M, from 2 M to 4 M, or from 3 M to 3.5 M, e.g., about 3.33 M.)

The invention provides methods for synthesizing hydrogel complexes in an efficient and scalable manner. For instance, hydrogel complexes can be synthesized using a spray apparatus (e.g., an apparatus including an atomizer).

In general, a polymer solution (e.g., an aqueous solution of alginic acid-PEG) can be injected into an atomizer simultaneously with a compressed gas (e.g., nitrogen) to atomize the polymer solution and produce a spray of droplets. This spray can be directed into a receiving solution to create the particles. The receiving solution may include a cation (e.g., a polycation, e.g., Ca²⁺), and, upon contact between the alginic acid-PEG droplets and the cationic receiving solution, alginic acid molecules within the droplets can become crosslinked by the cation, and hydrogel particles are formed. Additional components may be included as part of the receiving solution. For example, the receiving solution may include isopropyl alcohol, e.g., to further harden the particles during and/or after cationic crosslinking. Additionally or alternatively, the receiving solution may include other stabilizers and/or surfactants known in the art, as required.

Desired particle size can be achieved by tuning various parameters of the methods of the present invention. Generally, particle size (and, e.g., resulting hydrogel complex size) is related to the size of the droplets formed in the atomized spray (e.g., particle size is proportional to (e.g., directly proportional to) or slightly less than the size of the droplets formed in the atomized spray). Absent external variables, droplet size (and resulting particle or complex size) tends to decrease with (i) increasing gas pressure or gas flow rate through the atomizer, (ii) decreasing volumetric percentage of liquid flowing through the atomizer, (iii) decreasing viscosity of the polymer solution, and (iv) increasing spray angle.

In some embodiments, hydrogel particles are formed using an atomizer that sprays at a volumetric percentage of liquid from 30% to 90% (e.g., 35% to 80%, 40% to 75%, 45% to 70%, 50% to 65%, or 55% to 60% liquid droplets by volume, e.g., 35% to 40%, 40% to 45%, 45% to 50%, 50% to 55%, 55% to 60%, 60% to 65%, 65% to 70%, 70% to 75%, or 75% to 80% liquid droplets by volume). At these volume fractions, the size of resulting hydrogel particles will depend on other factors discussed above, but can range from 10 nm to 1,000 μm in mean diameter. In some embodiments, the volumetric percentages of liquid to gas identified herein will result in microparticles (e.g., particles having a diameter from 1.0 μm to 1,000 μm, e.g., from 1.0 μm to 500 μm, from 1.0 μm to 200 μm, from 1.0 μm to 100 μm, from 1.0 to 50 μm, from 1.0 μm to 25 μm, from 1.0 μm to 20 μm, from 2.0 μm to 20 μm, from 2.0 to 15 μm, from 2.0 μm to 10 μm, or from 2.0 μm to 5 μm). In other instances, the volumetric percentages of liquid to gas identified herein will result in nanoparticles (e.g., particles having a diameter from 1.0 nm to 1,000 nm, e.g., from 5 nm to 800 nm, from 10 nm to 600 nm, from 15 nm to 500 nm, from 20 nm to 400 nm, from 25 nm to 300 nm, from 50 nm to 250 nm, or from 100 nm to 200 nm, e.g., from 1 nm to 50 nm, from 50 nm to 100 nm, from 100 nm to 200 nm, from 200 nm to 300 nm, from 300 nm to 400 nm, from 400 nm to 500 nm, from 500 nm to 600 nm, from 600 nm to 700 nm, from 700 nm to 800 nm, from 800 nm to 900 nm, or from 900 nm to 1,000 nm).

The volumetric percentage of liquid (e.g., copolymer solution) to gas (e.g., nitrogen) is a function of the flow rate through the atomizer of the liquid relative to the gas. Accordingly, increasing the flow rate of gas relative to liquid through an atomizer will tend to result in smaller droplet size (and, e.g., smaller particle size). Gas flow rates can be controlled by pressurized reservoirs. In many embodiments, a gas source is a pressurized tank, and the rate of gas flow into the atomizer is controlled by a pressure control and/or regulator valve. Alternatively, gas may flow through an atomizer by other means, such as pumps (e.g., pressure-controlled pumps). Any gas reservoir providing a flow (e.g., a substantially constant flow) into the atomizer is suitable for use as part of the present invention.

Similarly, a liquid (e.g., a polymer solution) can be passed through the atomizer using a pressurized reservoir, such as a syringe pump (e.g., a syringe pump set to a constant flow rate). Alternatively, other pump formats can be used to drive liquid flow (e.g., polymer solution flow), such as peristaltic pumps. Systems such as peristaltic pumps provide the benefit of scalability, by enabling large volumes of liquid processing over extended durations. However, any liquid reservoir providing a flow (e.g., a substantially constant flow) into the atomizer is suitable for use as part of the invention.

In some embodiments of the invention, the atomizer is configured such that liquid flow and gas flow can be controlled independently. Such atomizers include external mix atomizers, in which the gas and liquid streams exit the atomizer nozzle at separate points. External mix atomizers enable an average atomized droplet size (and, e.g., the resulting average particle size) to be increased or decreased, for example, by decreasing or increasing, respectively, the gas flow rate, while holding the liquid flow rate constant.

Alternatively, an internal mix atomizer may be used as part of the invention. Internal mix atomizers introduce the gas to the liquid within the nozzle.

Further control over the volumetric percentage of liquid (e.g., polymer solution) to gas (e.g., nitrogen) can be maintained, for example, by attaching the liquid reservoir (e.g., syringe pump) to the atomizer using a ball valve to permit liquid flow upon liquid pressurization, thereby preventing variability in volumetric percentage at the start and finish of the atomization process.

Polymer solutions of the invention can be viscous (e.g., of a greater viscosity than water, i.e., greater than approximately 0.9-1 centipoise (cP) at 20-25° C.). Viscosity will depend on the concentration of the polymer (e.g., alginic acid-PEG copolymer) and the temperature. A liquid's viscosity is positively correlated with atomized droplet size (and, e.g., resulting particle size). The relationship between an atomized droplet size of a viscous solution and a corresponding atomized droplet of water can be estimated as:

D_{f}≅DₓV_{f}^{0.2}

where D_{f}=the droplet size of the viscous liquid, D_{w}=the droplet size of water, and V_{f}=the viscosity (cP) of the viscous liquid.

It will be understood that, in the case of a non-Newtonian fluid, viscosity varies with shear rate. For example, a copolymer solution containing alginic acid and/or PEG may exhibit shear thinning behavior, such that its viscosity decreases with increasing shear rate. In some embodiments, the apparent viscosity of a copolymer solution decreases by greater than 50% upon exposure of shear forces within an atomizer (e.g., by greater than 60%, 70%, 80%, 90%, 95%, 96%, 97%, 98%, or 99% upon exposure to shear forces within an atomizer). Thus, in evaluating the effect of viscosity on hydrogel particle size, a copolymer solution's apparent viscosity within the atomizer orifice should be considered. Factors that influence the apparent viscosity of a non-Newtonian fluid under a given shear rate (e.g., within an atomizer) are known in the art and can be calculated and/or empirically tested by known methods.

The spray angle of an atomizer is yet another parameter that influences droplet size. In general, a wider spray angle correlates with a smaller resulting droplet size. The present invention provides a means to synthesize microparticles (e.g., particles having a diameter from 1.0 μm to 1,000 μm, e.g., from 1.0 μm to 500 μm, from 1.0 μm to 200 μm, from 1.0 μm to 100 μm, from 1.0 to 50 μm, from 1.0 μm to 25 μm, from 1.0 μm to 20 μm, from 2.0 μm to 20 μm, from 2.0 to 15 μm, from 2.0 μm to 10 μm, or from 2.0 μm to 5 μm) using a spray angle from 1° to 50° (e.g., from 5° to 40°, from 10° to 30°, or from 15° to 25°, e.g., from 1° to 5°, from 5° to 10°, from 10° to 15°, from 15° to 20°, from 20° to 25°, from 25° to 30°, from 30° to 35°, from 35° to 40°, from 40° to 45°, or from 45° to 50°. Alternatively, nanoparticles, (e.g., particles having a diameter from 1.0 nm to 1,000 nm, e.g., from 5 nm to 800 nm, from 10 nm to 600 nm, from 15 nm to 500 nm, from 20 nm to 400 nm, from 25 nm to 300 nm, from 50 nm to 250 nm, or from 100 nm to 200 nm, e.g., from 1 nm to 50 nm, from 50 nm to 100 nm, from 100 nm to 200 nm, from 200 nm to 300 nm, from 300 nm to 400 nm, from 400 nm to 500 nm, from 500 nm to 600 nm, from 600 nm to 700 nm, from 700 nm to 800 nm, from 800 nm to 900 nm, or from 900 nm to 1,000 nm) can be synthesized using a spray angle from 50° to 150° (e.g., from 60° to 140°, from 70° to 130°, from 80° to 120°, or from 90° to 110°, e.g., from 50° to 60°, from 60° to 70°, from 70° to 80°, from 80° to 90°, from 90° to 100°, from 100° to 110°, from 110° to 120°, from 120° to 130°, from 130° to 140°, or from 140° to 150°).

In some embodiments, the atomizer produces a round spray pattern, which creates a conical, semi-conical, dome-shaped, or semi-cylindrical spray shape, depending on the spray angle, flow velocity, and droplet size. Thus, the spray angle is positively correlated with the total volume of the atomized spray. In some embodiments of the invention, the atomizer sprays the copolymer droplets downward (e.g., into a receiving solution), and the spray angle dictates the width (e.g., diameter) of the spray. In some embodiments, the width of the spray at the surface of the receiving solution is from 1.0 cm to 1,000 cm (e.g., from 2.0 cm to 100 cm, from 3.0 cm to 80 cm, from 5 cm to 70 cm, from 10 cm to 60 cm, from 20 cm to 50 cm, or from 20 cm to 40 cm, e.g., from 1.0 cm to 2.0 cm, from 2.0 cm to 3.0 cm, from 3.0 cm to 4.0 cm, from 5.0 cm to 6.0 cm, from 6.0 cm to 7.0 cm, from 7.0 cm to 8.0 cm, from 8.0 cm to 9.0 cm, from 10 cm to 15 cm, from 15 cm to 20 cm, from 20 cm to 30 cm, from 40 cm to 50 cm, from 50 cm to 60 cm, from 60 cm to 70 cm, from 70 cm to 80 cm, from 80 cm to 90 cm, from 90 cm to 100 cm, or greater).

In some embodiments, the atomizer is positioned at a height of 5 cm to 1,000 cm above the surface of the receiving solution (e.g., from 10 cm to 100 cm, from 15 cm to 85 cm, from 20 cm to 80 cm, from 25 cm to 75 cm, from 30 cm to 70 cm, from 35 cm to 65 cm, or from 40 cm to 60 cm, e.g., from 5 cm to 10 cm, from 10 cm to 15 cm, from 15 cm to 20 cm, from 20 cm to 25 cm, from 25 cm to 30 cm, from 30 cm to 40 cm, from 40 cm, to 50 cm, from 50 cm to 60 cm, from 60 cm to 70 cm, from 70 cm to 80 cm, from 80 cm to 90 cm, from 90 cm to 100 cm, or greater) above the surface of the receiving solution.

Given the above heights, widths (e.g., diameters), and shapes of the spray, the total volume of the spray can be approximated as the corresponding volume of a cone, a cylinder, or a value therebetween. Thus, the present invention includes conical total spray volumes from 1.3 cm³ to 1000 m³ (e.g., from 2 cm³ to 100 m³, from 5 cm³ to 10 m³, from 10 cm³ to 5 m³, from 50 cm³ to 1 m³, from 100 cm³ to 0.1 m³, or from 1,000 cm³ to 0.01 m³), semi-cylindrical spray volumes from 3 cm³ to 3000 m³ (e.g., from 2 cm³ to 100 m³, from 5 cm³ to 10 m³, from 10 cm³ to 5 m³, from 50 cm³ to 1 m³, from 100 cm³ to 0.1 m³, or from 1,000 cm³ to 0.01 m³), and any volume between that of the conical and cylindrical shapes from which the above values are derived.

The invention features a spray apparatus to accommodate any one or more of the above configurations. In some embodiments, the invention features a modular spray apparatus that can be modified to accommodate, for example, various distances from the atomizer to the receiving solution and/or receiving reservoir. In this case, the spray apparatus includes a jack rig assembly to enable a user to vary the total volume of the spray (e.g., by moving the receiving solution up or down, as necessary, relative to the atomizer).

After hydrogel particle synthesis, particles can be filtered using standard methods (e.g., sterile filtration methods, e.g., using a 140 μm nylon filter). Particle suspensions can otherwise by concentrated or purified, e.g., by standard centrifugation/resuspension methods.

Additionally or alternatively, hydrogel particles and/or complexes of the invention can be synthesized using methods currently known in the art. For example, hydrogel complexes can be formulated as microparticles by conventional microemulsion processes. Methods for synthesizing alginate beads for various applications are known in the art (see, for example, Hatch, et al., Langmuir 27 (2011): 4257-4264 and Sosnik, ISRN Pharmaceutics (2014):1-17).

Binding moiety conjugation (e.g., antibody conjugation) can be carried out using standard conjugation techniques including maleimide/thiol and EDC/NHS linking. Other useful conjugation methods are described in Hermanson et al., (2013) Bioconjugate Techniques: Academic Press. In some embodiments, binding moiety conjugation occurs immediately following particle formation, e.g., as described above. Alternatively, particles are stored (e.g., as frozen suspensions or lyophilized powders) prior to conjugation with binding moieties, and binding moiety conjugation is performed prior to cell treatment (e.g., immediately prior to cell treatment, with or without further purification, e.g., filtration and/or centrifugation/resuspension).

### Methods of Use

The invention features a method of modulating immune cells, e.g., expanding a population of T cells, using the particle or hydrogel complex described above. Prior to modulation, e.g., expansion, a source of immune cells, e.g., T cells, can be obtained from a subject or alternative source (e.g., a frozen cell stock or cell line). Immune cells, e.g., T cells, can be obtained from a number of sources, including peripheral blood mononuclear cells (PBMCs), bone marrow, lymph node tissue, spleen tissue, tumors, or frozen stocks of allogenic or autologous cells. In certain embodiments of the present invention, an immune cell, e.g., T cell, line available in the art may be used. Immune cells, e.g., T cells, can also be obtained from a unit of blood collected from a subject using any number of techniques known to the skilled artisan, such as Ficoll separation or through a PERCOLL® gradient. Cells from the circulating blood of a subject (e.g., PBMCs) can be obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. Immune cells, e.g., T cells, can be enriched prior to treatment with the complex of the present invention through conventional techniques such as magnetic bead negative selection or fluorescence activated cell sorting (FACS). Immune cells, e.g., T cells, can be antigen-specific, antigen-nonspecific, or tumor specific. They can include populations of CD4⁺ T cells, CD8⁺ T cells, NK T cells, or, for autoimmune or transplant rejection therapies, regulatory T cells. They may include populations of regulatory T cells, NK cells, NK T cells, CIK cells, TIL cells, HS cells (undifferentiated and differentiated), MS cells (undifferentiated and differentiated), iPS cells (undifferentiated and differentiated), or ES cells (undifferentiated and differentiated).

In addition to isolation steps, immune cells, e.g., T cells, that have been obtained from a subject may be further processed prior to or after incubation with the complex of the present invention. For example, cells can undergo genetic engineering to equip them with certain functional characteristics, such as in the process of chimeric antigen receptor (CAR) engineering, which equips a patient's cells to recognize cancer antigens. In this case, the CAR engineering procedure may be performed prior to treatment with the present complexes in order to expand the initially small number of CAR T cells into a substantial activated population. Other genetic procedures used for adoptive therapy are discussed in Rosenburg et al. (Nat Rev Cancer 8 (2008): 299-308). In other embodiments, such as those not involving genetic modification, such as bispecific T cell engager (BITE) technology (see, for example, WO 2011/057124), procedures may be performed after the cells have been expanded using the present invention. Other non-genetic processing procedures include but are not limited to treatment with IL-2, IL-4, IL-7, IL-10, IL-12, IL-15, IL-21, or TGF-β.

### Sample Treatment

In one aspect of the invention, the starting immune cell population, e.g., T cell population, is incubated with the complexes. The ratio of complexes to starting cell number will depend on the size and shape of the complex. For this purpose, it will be understood by a person of skill in the art that the ratio of surface area between target immune cells, e.g., T cells, and complexes is a significant factor governing the degree of resulting immune cell, e.g., T cell, expansion and/or activation. In some embodiments, the surface area ratio between target cells and particulate complexes (e.g., microparticle complexes, e.g., complexes having a mean diameter between 1 μm and 100 μm, e.g., about 10 μm) is between about 1:100 and about 100:1 (e.g., about 1:100, about 1:80, about 1:50, about 1:25, about 1:10, about 1:5, about 1:4, about 1:3, about 1:2, about 1:1, about 2:1, about 3:1, about 4:1, about 5:1, about 10:1, about 25:1, about 50:1, about 80:1, or about 100:1). In some embodiments, the relative quantity of particle complexes to cells is measured by relative number of complexes to cells. For example, in an initial cell culture containing 1.2×10⁶ T cells per mL, or an initial cell culture having a volume of about 400 mL, the initial number of particulate complexes (e.g., microparticle complexes, e.g., complexes having a mean diameter between 1 μm and 100 μm, e.g., about 10 μm) can be from 0.1×10⁶ to 20×10⁶, from 0.5×10⁶ to 10×10⁶, from 1×10⁶ to 6×10⁶. In some embodiments, the complexes may be measured by mass. For example, in an initial cell culture containing 1.2×10⁶ T cells per mL, or an initial cell culture having a volume of about 400 mL, the initial mass of complexes (e.g., microparticle complexes, e.g., complexes having a mean diameter between 1 μm and 100 μm, e.g., about 10 μm) can be from 1.0 μg/mL to 1.0 mg/mL (e.g., from 2.0 μg/mL to 800 μg/mL, from 5.0 μg/mL to 500 μg/mL, from 10 μg/mL to 400 μg/mL, from 20 μg/mL to 300 μg/mL, from 50 μg/mL to 250 μg/mL, e.g., from 1.0 μg/mL to 5.0 μg/mL, from 5.0 μg/mL to 10 μg/mL, from 10 μg/mL to 20 μg/mL, from 20 μg/mL to 30 μg/mL, from 30 μg/mL to 40 μg/mL, from 40 μg/mL to 50 μg/mL, from 50 μg/mL to 60 μg/mL, from 60 μg/mL to 75 μg/mL, from 75 μg/mL to 100 μg/mL, from 100 μg/mL to 200 μg/mL, from 200 μg/mL to 250 μg/mL, from 250 μg/mL to 500 μg/mL, from 500 μg/mL to 750 μg/mL, from 750 μg/mL to 1.0 mg/mL, from 1.0 mg/mL to 1.5 mg/mL, or from 1.5 mg/mL to 2.0 mg/mL. It will be understood that any of the above referenced quantities is scalable with cell concentration within the culture. Other factors which should be considered when incubating starting immune cells, e.g., T cells, with complexes are the density of binding moieties on the complex surfaces and the specific binding moieties chosen (e.g., their binding affinity, or EC₅₀, receptor concentration on immune cells, e.g., T cells). It will be appreciated that, as immune cells, e.g., T cells, proliferate over the course of multiple days, their volumetric requirements will increase, which may limit the total volume that can be allocated to complexes.

### Incubation Procedures

Methods of the present invention include incubating immune cells, e.g., T cells, with complexes in a suitable vessel. Such cell-culture vessels are known in the art and can include cell culture plates, flasks, or bioreactors of any suitable size. The type or size of vessel may be altered as the cell population expands (e.g., from a 6-well plate to a T25 flask to a T75 flask). Cell culture vessels are preferably sterile, and may be configured for optimal gas exchange or media exchange, such as perfusion capable systems, which are known in the art. A population of cells containing immune cells, e.g., T cells can be seeded at any suitable concentration for induction of expansion, as known in the art. For example, cells can be seeded at a concentration of between about 0.2×10⁶ and 10×10⁶ cells/ml (e.g., between 0.5×10⁶ and 1.5×10⁶, e.g., about 0.5×10⁶, or about 1.2×10⁶ cells/ml).

Complexes may require special ionic conditions, e.g., to maintain a solid structure in solution. For example, cell culture media can be supplemented with ions, such as cations, e.g., polycations, e.g., Ca²⁺, through addition of salts, e.g., CaCl₂). Ions can be present at any physiologically suitable concentration (e.g., 1.0 nM to 100 mM, e.g., 1.0 μM to 10 mM, e.g., 0.1 mM to 10 mM, e.g., 0.1 mM, 0.2 mM, 0.5 mM, 1.0 mM, 2 mM, 3 mM, 4 mM, 5 mM, 6 mM, 7 mM, 8 mM, 9 mM, or 10 mM).

Additional factors can be included in an immune cell, e.g., a T cell, expansion media. Such factors include small molecule, peptide, and protein factors known in the art, e.g., vitamins, amino acids, cytokines, or growth factors. Cytokines known to support immune cell, e.g., T cell, expansion include interleukin-1 (IL-1), IL-2, IL-4, IL-7, IL-15, IL-18, IL-21, IL-23, and interferon-gamma (IFN-γ). Small molecule factors that may be included in the expansion media include mTOR inhibitors (e.g., rapamycin) and AKT inhibitors (e.g., AKT Inhibitor VIII).

### Cell Expansion

Ex vivo immune cell, e.g., T cell, expansion protocols are well-developed, especially for human samples, and can often yield cell number increases in the hundred-fold range, over the course of multiple weeks of culture. Thus, multiple rounds of expansion may be required to overcome constraints on physical space and media nutrient depletion. To accommodate these constraints, the complexes of the present invention can be dissolved and reapplied multiple times (e.g., once, twice, or 3-12 times) over the course of an immune cell, e.g., a T cell expansion protocol (e.g., over the course of 1 week to 6 weeks, e.g., over the course of 1 to 2 weeks, 2 to 3 weeks, or 3 to 4 weeks, e.g., over the course of 7 days, 8 days, 9 days, 10 days, 12 days, 14 days, 16 days, 18 days, 20 days, 21 days, or more). Such dissolution/re-application cycles can be achieved by washing out cation chelators from the media by centrifugation or other known methods after each dissolution. Alternatively, additional complexes can be introduced into the cultures without removing separation units of the existing complexes, thus bypassing the need for media changes.

In one aspect of the present invention, the immune cell, e.g., T cell, expansion protocol proceeds for a predetermined length of time that is suitable to generate a desired number of T cells, a representative phenotype, or both. For example, methods of the invention can be used to expand the number of starting cells (e.g., T cells, e.g., CD4⁺ T cells and/or CD8⁺ T cells) from 1-fold to 1,000,000-fold or greater (e.g., greater than 10-fold, greater than 100-fold, greater than 1,000-fold, greater than 10,000-fold, greater than 100,000-fold, or greater than 1,000,000-fold, e.g., from 1-fold to 10-fold, from 10-fold to 100-fold, from 100-fold to 1,000-fold, from 1,000-fold to 10,000-fold, from 10,000-fold to 100,000-fold, or from 100,000-fold to 1,000,000-fold). In some embodiments, the starting population is expanded from 100-fold to 1,000-fold over 9 days (e.g., from 200-fold to 400-fold, or from 300-fold to 350-fold).

The phenotypic properties of immune cell population, e.g., T-cell populations, of the present invention can be monitored by a variety of methods, and the isolation can be performed after the desired phenotype is acquired. Relevant phenotypes are metabolic changes such as biochemical or morphological changes (e.g., change in frequency of cell division, change in cytokine expression profile, change in cell diameter (e.g., median cell diameter), change in surface molecule expression, or change in cellular motility). Assays for monitoring such changes include standard flow cytometry methods, ELISA, microscopy, migration assays, metabolic assays, and other techniques known by those skilled in the art. A phenotype of a resulting cell can be determined relative to a reference cell or a reference population. In the case of an expression level of a marker determined by flow cytometry, for example, a reference population may be a population of cells that are unstained or stained with an isotype antibody (e.g., a “fluorescence minus one” control).

Methods of the invention provide for expansion of a population of cells containing immune cells, e.g., CD4⁺ T cells and/or CD8⁺ T cells. In some embodiments, one or more properties of the hydrogel complex (e.g., stiffness, hydrophilicity, density and/or composition of binding moieties) influences the phenotype of the cells as they expand, as discussed previously. For example, by culturing a starting population of immune cells, e.g., T cells, with a complex having a low-elastic modulus polymeric moiety (e.g., an alginic acid-based polymeric moiety having an elastic modulus between 100 pascals (Pa) and 100,000,000 Pa (e.g., from 100 Pa to 1,000 Pa, from 1,000 Pa to 10,000 Pa, between 10,000 Pa to 100,000 Pa, between 100,000 Pa and 1,000,000 Pa, 1,000,000 Pa and 10,000,000 Pa, or 10,000,000 Pa and 100,000,000 Pa, e.g., less than 1,000,000 Pa, less than 900,000 Pa, less than 800,000 Pa, less than 700,000 Pa, less than 600,000 Pa, less than 500,000 Pa, less than 400,000 Pa, less than 300,000 Pa, less than 200,000 Pa, less than 100,000 Pa, less than 50,000 Pa, or less than 10,000 Pa), at one or more points in time over the course of incubation with the cells) a resulting population of immune cells, e.g., T cells, may have a greater number or percentage of particular cells, e.g., CD8⁺ T cells, relative to a reference population (e.g., a starting population or a control population, e.g., using control beads or soluble factors). For example, the expanded population may contain more than 1.1-fold, more than 1.2-fold, more than 1.3-fold, more than 1.4-fold, more than 1.5-fold, more than 1.6-fold, more than 1.7-fold, more than 1.8-fold, more than 1.9-fold, more than 2-fold, more than 2.5-fold, more than 3-fold, more than 4-fold, more than 5-fold, more than 6-fold, more than 7-fold, more than 8-fold, more than 9-fold, more than 10-fold, more than 12-fold, more than 15-fold, more than 20-fold, more than 30-fold, more than 40-fold, more than 50-fold, or more than 100-fold the number of particular cells, e.g., CD8⁺ T cells, relative to its reference population (e.g., a starting population or a control population, e.g., using control beads or soluble factors). Similarly, a population of immune cells, e.g., T cells, expanded using the complexes of the invention may have a lower number or percentage of particular cells, e.g., CD4⁺ T cells, relative to the reference population (e.g., a starting population or a control population, e.g., using control beads or soluble factors). For example, the expanded population may contain more than 95% fewer, more than 90% fewer, more than 80% fewer, more than 70% fewer, more than 60% fewer, more than 50% fewer, more than 40% fewer, or more than 30% fewer particular cells, e.g., CD4⁺ T cells, relative to its reference population (e.g., a starting population or a control population, e.g., using control beads or soluble factors). Similarly, the expanded T cell population may have a greater cell ratio, e.g., CD8-to-CD4 T cell ratio, than the reference population. For example, the cell ratio, e.g., CD8-to-CD4 T cell ratio, in an expanded population may be more than 1.1-fold, more than 1.2-fold, more than 1.3-fold, more than 1.4-fold, more than 1.5-fold, more than 1.6-fold, more than 1.7-fold, more than 1.8-fold, more than 1.9-fold, more than 2-fold, more than 2.5-fold, more than 3-fold, more than 4-fold, more than 5-fold, more than 6-fold, more than 7-fold, more than 8-fold, more than 9-fold, more than 10-fold, more than 12-fold, more than 15-fold, more than 20-fold, more than 30-fold, more than 40-fold, more than 50-fold, or more than 100-fold greater relative to its reference population. In general, methods of the invention can be used to expand the immune cells, e.g., T cells, in a population, e.g., such that the resulting cell population (e.g., a population of mixed lymphocytes) contains more than 1.1-fold, more than 1.2-fold, more than 1.3-fold, more than 1.4-fold, more than 1.5-fold, more than 1.6-fold, more than 1.7-fold, more than 1.8-fold, more than 1.9-fold, more than 2-fold, more than 2.5-fold, more than 3-fold, more than 4-fold, more than 5-fold, more than 6-fold, more than 7-fold, more than 8-fold, more than 9-fold, more than 10-fold, more than 12-fold, more than 15-fold, more than 20-fold, more than 30-fold, more than 40-fold, more than 50-fold, or more than 100-fold the number of immune cells, e.g., T cells, relative to its reference population.

In some embodiments, the methods of the present invention allow for expansion of a population of immune cells containing naïve T cells, central memory T cells, and/or effector memory cells. The composition of the hydrogel can influence the percentage and/or total number of naïve T cells, central memory T cells, and/or effector memory cells present in a population (e.g., an expanded population). For example, by culturing a starting population of T cells with a complex having a low-elastic modulus polymeric moiety (e.g., an alginic acid-based polymeric moiety having an elastic modulus between 100 pascals (Pa) and 100,000,000 Pa (e.g., from 100 Pa to 1,000 Pa, from 1,000 Pa to 10,000 Pa, between 10,000 Pa to 100,000 Pa, between 100,000 Pa and 1,000,000 Pa, 1,000,000 Pa and 10,000,000 Pa, or 10,000,000 Pa and 100,000,000 Pa, e.g., less than 1,000,000 Pa, less than 900,000 Pa, less than 800,000 Pa, less than 700,000 Pa, less than 600,000 Pa, less than 500,000 Pa, less than 400,000 Pa, less than 300,000 Pa, less than 200,000 Pa, less than 100,000 Pa, less than 50,000 Pa, or less than 10,000 Pa), at one or more points in time over the course of incubation with the cells) a resulting population of T cells may include a high percentage of naïve T cells (e.g., naïve CD4⁺ T cells or naïve CD8⁺ T cells). In some instances, naïve T cells are characterized as having one or more properties (surface markers or secreted cytokines) of Table I (e.g., relative to a reference population).

TABLE I
Model of memory T cell subtype marker expression

| | | Secretion |
|---|---|---|
| T cell subtype | Surface marker expression profile | profile |
| Naïve T cells | CD45RA⁺ CD45RO⁻ CCR7⁺ CD62L⁺ | IL-4⁻ IFN-γ⁻ |
| Central memory | CD45RA⁻ CD45RO⁺ CCR7⁺ CD62L⁺ | IL-4⁻ IFN-γ⁻ |
| T cells | | |
| Effector memory | CD45RA⁻ CD45RO⁺ CCR7⁻ CD62L⁻ | IL-4⁺ IFN-γ⁺ |
| T cells | | |

Methods of using hydrogel complexes provided herein may also induce distinct activation marker expression profiles (e.g., expression profiled of CD25, CD69, and or other activation markers (e.g., surface markers and/or cytokine secretion)). For example, culturing a starting population of T cells with a complex having a low-elastic modulus polymeric moiety (e.g., an alginic acid-based polymeric moiety having an elastic modulus between 100 pascals (Pa) and 100,000,000 Pa (e.g., from 100 Pa to 1,000 Pa, from 1,000 Pa to 10,000 Pa, between 10,000 Pa to 100,000 Pa, between 100,000 Pa and 1,000,000 Pa, 1,000,000 Pa and 10,000,000 Pa, or 10,000,000 Pa and 100,000,000 Pa, e.g., less than 1,000,000 Pa, less than 900,000 Pa, less than 800,000 Pa, less than 700,000 Pa, less than 600,000 Pa, less than 500,000 Pa, less than 400,000 Pa, less than 300,000 Pa, less than 200,000 Pa, less than 100,000 Pa, less than 50,000 Pa, or less than 10,000 Pa), at one or more points in time over the course of incubation with the cells, can result in lower expression of activation marker expression (e.g., CD25 and/or CD69) relative to a reference population. For example, an activation marker may have lower relative expression at one or more time points along an expansion protocol (e.g., the peak expression of the activation marker), the activation marker may increase at a slower rate relative to a control group, or the activation marker may be downregulated (e.g., after initial expression or upregulation) to a greater extent relative to a control group.

In some embodiments of the methods described herein, hydrogel complex treatment induces CD25 upregulation on CD4⁺ T cells and/or CD8⁺ T cells a slower rate (e.g., less than 95% of the rate, less than 90% of the rate, less than 80% of the rate, less than 70% of the rate, less than 60% of the rate, less than 50% of the rate, less than 40% of the rate, or less than 30% of the rate) relative to a control group. In some embodiments, CD25 expression is decreased (e.g., at least 10% less, at least 20% less, at least 30% less, at least 40% less, at least 50% less, at least 60% less, at least 70% less, at least 80% less, or at least 90% less) relative to a control group, at any point along the expansion period (e.g., at the end of the expansion period).

Treatment with hydrogel complexes can also effect CD69 expression of CD4⁺ T cells and CD8⁺ T cells. In some embodiments, hydrogel complex treatment induces a slower rate of upregulation of CD69 (e.g., less than 95% of the rate, less than 90% of the rate, less than 80% of the rate, less than 70% of the rate, less than 60% of the rate, less than 50% of the rate, less than 40% of the rate, or less than 30% of the rate) relative to a control group. In some embodiments, CD69 expression is decreased (e.g., at least 10% less, at least 20% less, at least 30% less, at least 40% less, at least 50% less, at least 60% less, at least 70% less, at least 80% less, or at least 90% less) relative to a control group, at any point along the expansion period (e.g., at its peak expression or at the end of the expansion period). Additionally or alternatively, the peak expression of CD69 may be lower as a result of hydrogel complex treatment relative to a control group (e.g., less than 95%, less than 90%, less than 80%, less than 70%, less than 60%, less than 50%, less than 40%, less than 30%, or less than 20% of peak expression of the control group).

### Purification

In the presence of a cation, e.g., calcium, alginic acid is crosslinked and solid. Upon completion of immune cell (e.g., T cell) processing, the hydrogel can be dissolved (e.g., liquefied) by incubation in a release buffer containing a chelator to liquefy the complex and release the immune cell (e.g., T cell). The resulting immune cells (e.g., T cells) can thereby be cleaned of impurities and ready for infusion into a patient in need thereof.

EDTA is a well-characterized calcium chelator of use in the present invention. EDTA can be used at a concentration from 0.1 mM to 10 mm in the form of a physiologically inert buffer (e.g., at a concentration from 0.5 mM to 5 mM, from 0.7 mM to 3 mM, or from 1.0 mM to 2.0 mM, e.g., at a concentration of 1.0 mM, 1.5 mM, or 2.0 mM). A release buffer of the invention may also include, e.g., NaCl (e.g., at about 137 mM), KCl (e.g., at about 2.7 mM), Hepes (e.g., at about 25 mM) and/or Na₂H₂PO₄.2H₂O (e.g., at about 0.75 mM).

Cell culture media can be exchanged for the ion chelator solution, e.g., EDTA buffer, by centrifugation and subsequent resuspension of cell pellet, e.g., in EDTA buffer. Other methods may also be used. For example, a slow release may be achieved by controllably or gradually adding a low concentration of chelating agent and/or by removing Ca²⁺ supplementation. The cell/ion chelator suspension may also be agitated, e.g., by pipetting or vortexing for about 5 seconds. During this step, the hydrogel dissolves, and the cells are released. The isolated cells can then be returned to cell culture media or isotonic solution (e.g., for administration into a patient).

In certain embodiments, binding moieties, e.g., antibodies, that bind to immune cells (e.g., T cells) are contacted with the cells in the absence of complexes of the invention. Complexes that bind to the binding moieties bound to the T cells can then be added for incubation and/or expansion.

Alternative methods of dissolving a hydrogel are described herein and known in the art. For example, the hydrogel can be dissolved by change in temperature, change in pH, hydrolysis, oxidation, enzymatic degradation, or physical degradation.

### EXAMPLES

### Example 1. Spray Apparatus Assembly

A spray apparatus was prepared as follows. A 32-gallon tank (70 cm tall by 55 cm wide; RUBBERMAID® BRUTE® Round Container) was placed on a 15.5 cm level block, such that the distance from the top of the tank to the ground was 85.5 cm. The interior of the tank was sprayed and wiped down with 70% isopropyl alcohol. A jack rig was adjusted to hold a platform at a height of 20 cm and leveled using a spirit level. The jack rig was placed onto the four bolts in the bottom of the tank.

A spray reservoir matching the cross-section of the tank was sprayed and wiped down with 70% isopropyl alcohol. Prior to assembling the spray reservoir within the tank, 2.0 L of receiving solution was added thereto. The receiving solution was prepared by combining 1,000 mL of 70% isopropyl alcohol, 970 mL of sterile water, and 30 mL of 3.33 M CaCl₂, resulting in a 2 L volume of 35% isopropyl alcohol, 50 mM CaCl₂. The spray reservoir was then positioned onto the jack rig platform within the tank.

The spray nozzle mount was sprayed and wiped down with 70% isopropyl alcohol and placed on the top rim of the tank with its atomizer fittings face up. The spray nozzle mount was centered on the tank using pre-measured markings on the tank and the spray nozzle mount. The spray nozzle mount was fastened to the tank using 2¾-inch screws.

A round spray atomizer (XA ER 050 A; BETE Fog Nozzle, Inc.) was attached at its liquid intake side to a ball valve connected to a 30-mL syringe via TYGON® tubing. 10 mL of 70% isopropyl alcohol, 10 mL water, and 30 mL or air were sequentially transferred into the syringe and flushed through the atomizer.

The air intake side of the atomizer was attached via tubing to a compressed nitrogen tank regulator valve, and the atomizer was placed within fittings on the spray nozzle mount. The compressed nitrogen tubing was secured to the spray nozzle mount using 2¾-inch screws, taking care not to overtighten the screws to avoid compressing the tubing. The atomizer was secured within the fittings by stretching a rubber band from one screw to the other, over the atomizer.

The compressed nitrogen tank valve was open to set the pressure to 50 psi, at which point the regulator valve was closed.

The tank cover was sprayed and wiped down with 70% isopropyl alcohol and positioned on the top rim of the tank such that cover openings aligned with both ends of the tubing. Rubber bands were used to secure the tank cover in place, using screwed affixed to the sides of the tank and the top of the tank cover.

### Example 2. Synthesis of Hydrogel Complexes

Alginic acid-PEG copolymer was first synthesized by conjugating sodium alginate to 4-arm PEG amine. Sodium alginate and 4-arm PEG amine were dissolved in water at a ratio of 2:1 sodium alginate-to-PEG-amine (22.5 mg/mL sodium alginate and 11.25 mg/mL PEG-amine), followed by addition of EDC (0.4 mg/mL) and sulfo-NHS (1.1 mg/mL). The PEG-alginate conjugation reaction occurred overnight at room temperature.

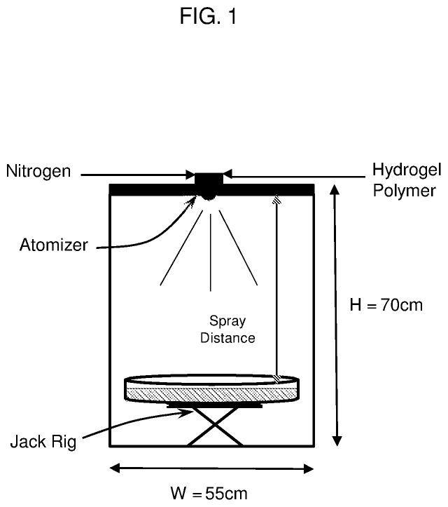

Hydrogel complexes were formed into microparticles using the spray apparatus, prepared as in Example 1 and illustrated in FIG. 1. In general, the spray apparatus included a syringe pump to inject the copolymer solution into an atomizer, which directs the solution, under pressure from a gas cylinder, as an aerosol into a spray receptacle containing a cationic liquid, within which the hydrogel solidifies into particles.

The syringe was loaded with the polymer solution prepared above. The regulator valve was opened to induce flow of nitrogen from the compressed nitrogen tank at a pressure of 50 psi. The polymer solution was injected into the atomizer automatically using a syringe pump set to a flow rate of 10 mL per minute. The atomizer sprayed the atomized spray downward, perpendicularly relative to each intake valve, in a spray radius of about 60 mm. The spray contacted the receiving solution in the spray reservoir under mixing, and the hydrogel particles were solidified.

The resulting suspension of hydrogel particles was transferred from the spray reservoir to a gravity filtration system (STERIFIL® Aseptic System and Holder; 140 μm nylon filter) and collected in a 1 L Erlenmeyer flask. Hydrogel particles were concentrated by centrifugation and resuspended in HEPES buffered saline (HBS).

Antibodies were conjugated to the hydrogel particles immediately prior to cell culture. Anti-CD3 (clone OKT3) and anti-CD28 (clone 28.2) were incubated with microparticles in HBS containing standard concentrations of EDC and sulfo-NHS to conjugate to the surface of the alginic acid-PEG hydrogel microparticles.

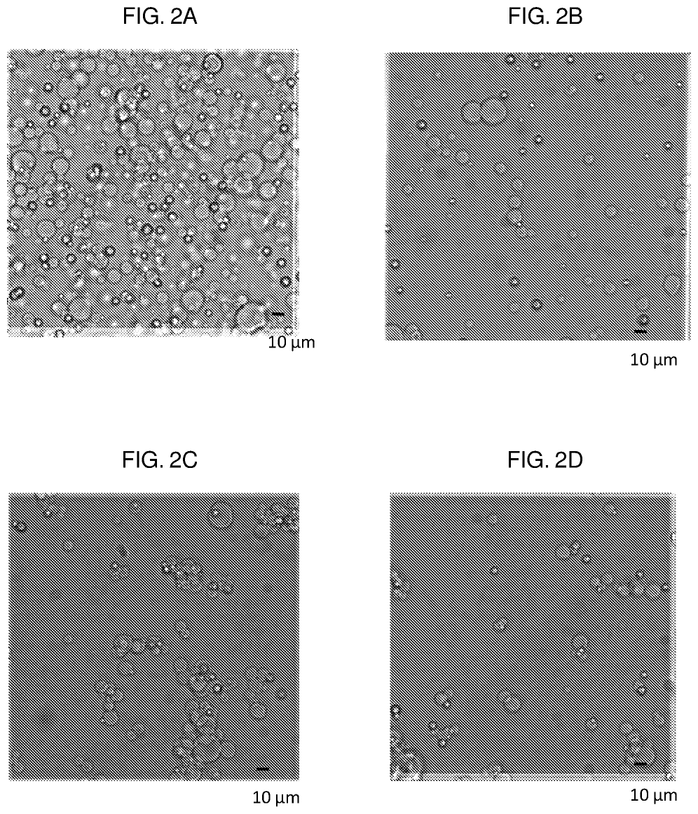

To assess the size and morphology distributions of the complexes, the suspension was visualized under a microscope at high and low concentrations, as shown in FIGS. 2A-2D. The complexes were then washed by centrifugation and stored HBS containing 20 mM Ca²⁺, pH 7.4.

### Example 3. Expansion of T Cells Using Anti-CD3/Anti-CD28 Hydrogel Complexes

T cells were purified from human peripheral blood by selection for CD3 expression using conventional methods. T cells were seeded in a 24-well plate at 0.5×10⁶ cells per 400 mL medium (RPMI supplemented with fetal bovine serum (FBS), GLUTAMAX™, HEPES, 15 ng/mL IL-2, and 2 mM CaCl₂)) per well. Hydrogel complexes generated as described in Example 2 were added at complex-to-cell ratios of 5:1 or 10:1, and each complex/cell suspension was mixed by gentle agitation. Control groups included unstimulated cells and cells treated with control anti-CD3/anti-CD28 beads at a bead-to-cell ratio of 3:1, according to the manufacturer's instructions.

Over the course of cell proliferation, every 2-3 days as needed, the cell culture was transferred to larger vessels and supplemented with fresh medium. In particular, the culture was transferred from the 24-well plate to a 6-well plate, from the 6-well plate to a T25 flask, and from the T25 flask to a T75 flask.

After 9 days, cell cultures were centrifuged and supernatant was discarded, and cells and complexes were resuspended in buffer containing 1 mM EDTA. Agitation of this suspension dissolved the complexes, and the cells were washed by centrifugation.

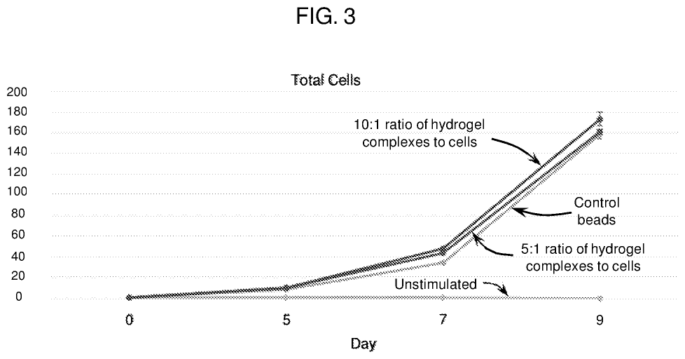

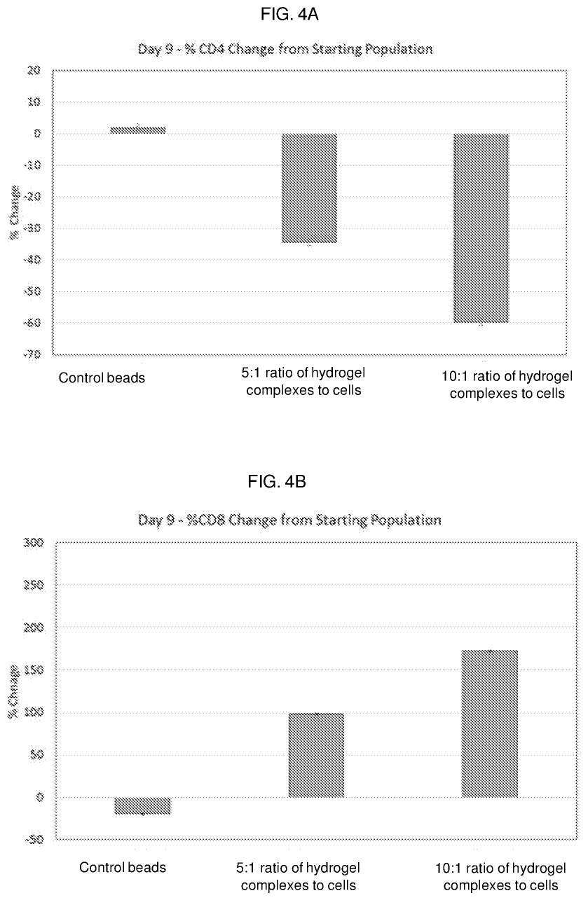

Over the course of expansion, the total number of cells was counted. As shown in FIG. 3, hydrogel complexes induced T cell proliferation to a similar or greater degree than control beads (i.e., from 0.5×10⁶ cells to between 160×10⁶ cells and 180×10⁶ cells, i.e., from about 320-fold to about 360-fold). Importantly, T cells expanded by the hydrogel complexes included a significantly greater number and frequency of CD8⁺ cells, relative to control beads, as shown in FIGS. 4A and 4B. FIG. 4A shows that, while control beads had little effect on the percentage of CD4⁺ cells in the expanded population after 9 days of treatment, hydrogel complexes reduced the relative number of CD4⁺ cells by greater than 30% at a 5:1 complex-to-cell ratio and by about 60% at a 10:1 complex-to-cell ratio. Conversely, FIG. 4B shows that the percentage of CD8⁺ cells in the expanded population increased by about 100% at a 5:1 complex-to-cell ratio and by nearly 175% at a 10:1 complex-to-cell ratio, while CD8⁺ frequency slightly decreased as a result of expansion with control beads. Thus, hydrogel complexes met or exceeded the expansion of control beads, while skewing expanded T cell phenotype to cytotoxic CD8⁺ cells.

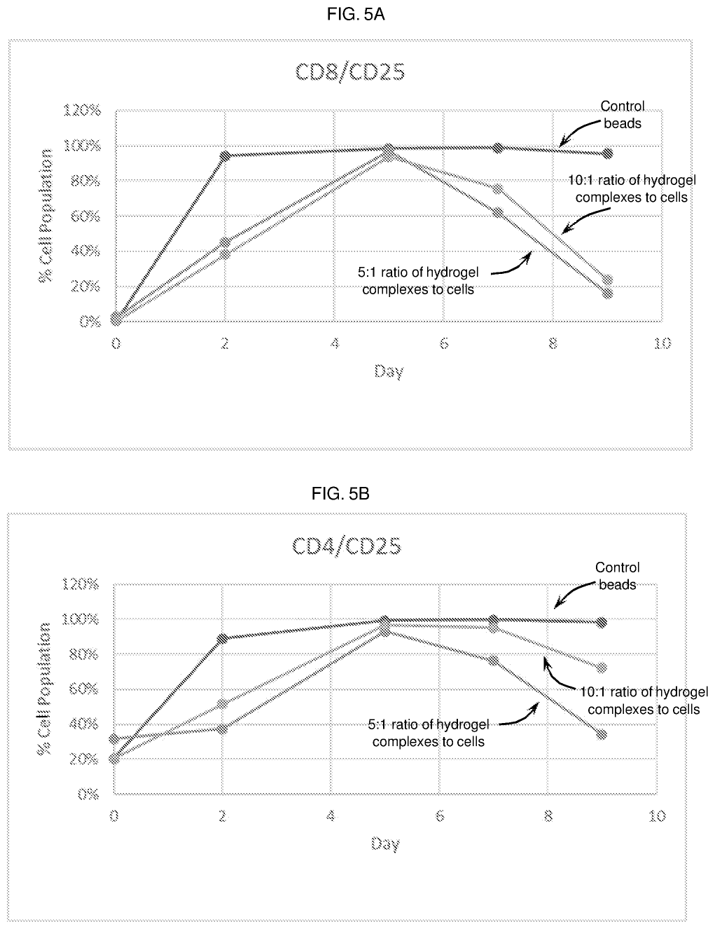

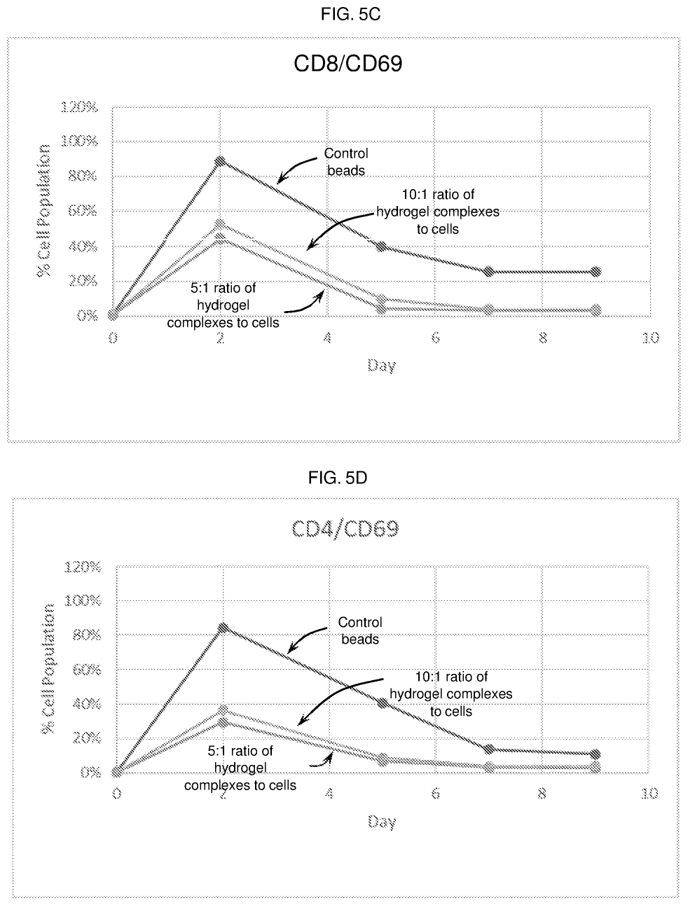

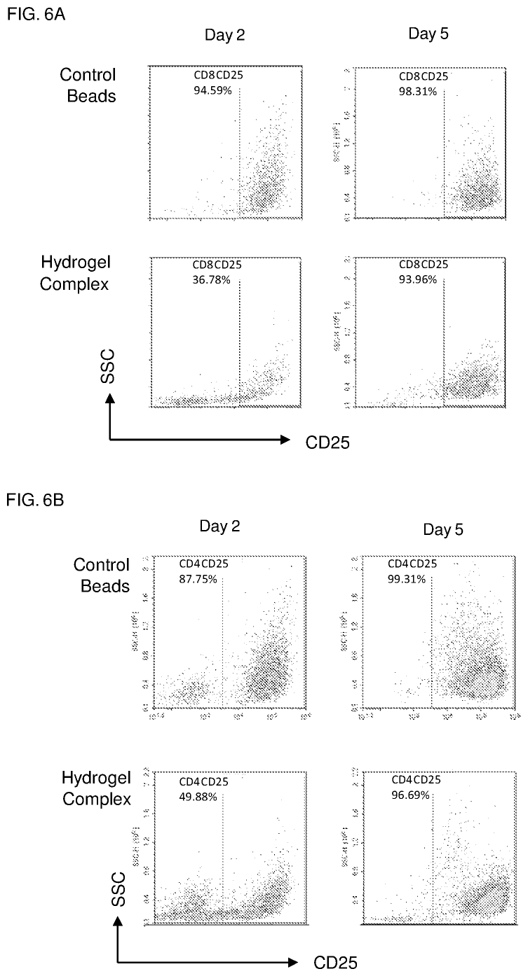

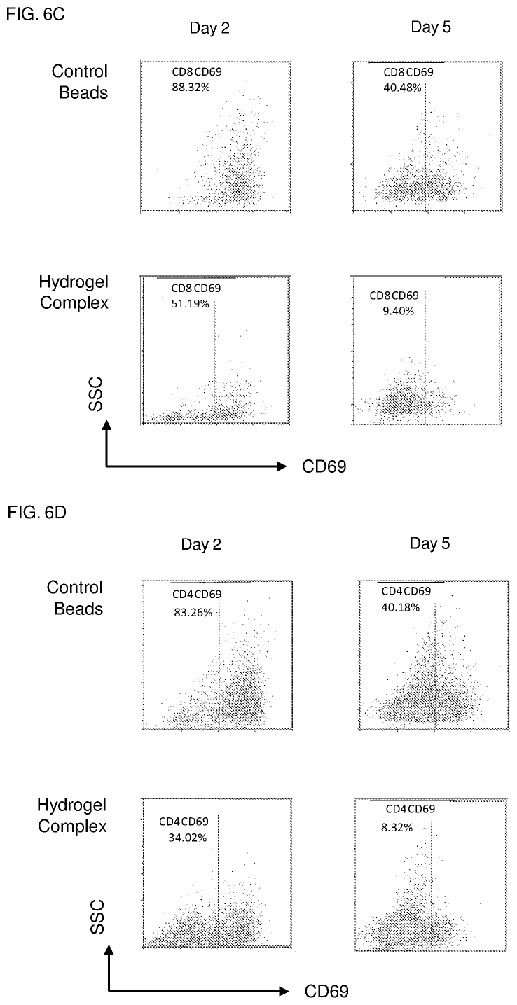

Activation markers expressed on CD8⁺ T cells and CD4⁺ T cells over the course of treatment are shown in FIGS. 5A-5D. Expression profiles of CD25 and CD69 were different as a result of treatment with hydrogel complexes in comparison to control beads. In particular, hydrogel complexes induced a gradual increase in CD25 expression in both CD8⁺ T cells and CD4⁺ T cells, which peaked on day 5. In contrast, control beads triggered a rapid increase of CD25 expression in both CD8⁺ T cells and CD4⁺ T cells, which approached 100% expression at day 2 and was maintained throughout the 9-day culture. (FIGS. 5A and 5B). FIGS. 5C and 5D show the kinetics of CD69 expression, which was upregulated to a lesser extent in hydrogel complex-treated cells than in control bead-treated cells, and decreased in all treatment groups between days 2 and 5 of culture. For each cell type, the 10:1 hydrogel complex-to-cell ratio induced a slightly higher expression of CD25 and CD69, relative to the 5:1 hydrogel complex-to-cell ratio. FIGS. 6A-6D show the flow cytometry graphs corresponding with control beads and hydrogel complexes at a 10:1 complex-to-cell ratio at days 2 and 8, as shown in FIGS. 5A-5D, respectively.

### Example 4. Modulation of Ligand and Ligand Density Modulates T Cell Expansion

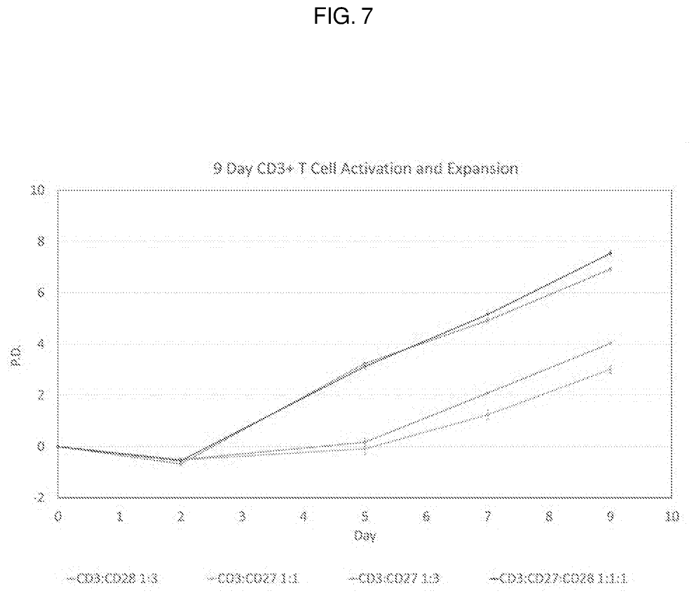

Primary human CD3+T lymphocytes were seeded at a density of 1×10⁶ cells/mL and cultured in advanced RPMI medium supplemented with fetal bovine serum, glutamate, HEPES, and recombinant human IL-2. On day 0, cells were stimulated with an equal number of hydrogel complexes conjugated with various ratios of antibodies (anti-CD3, anti-CD27, or anti-CD28 antibodies in the ratios shown in FIG. 7). Cell expansion is presented as population doublings (P.D.) and indicates the influence of ligand and ligand density upon T cell growth.

### Example 5. Modulation of Ligand and Ligand Density Modulates T Cell Phenotype

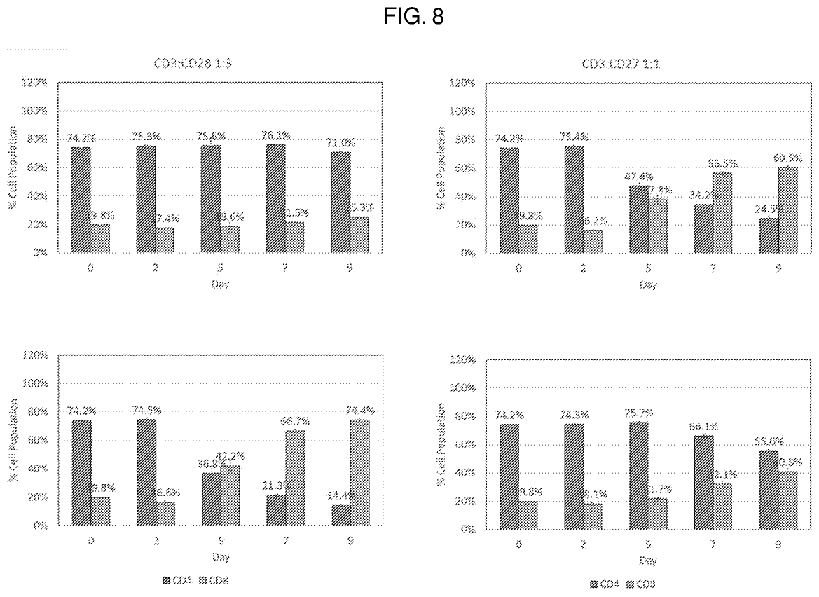

Primary human CD3+T lymphocytes were seeded at a density of 1×10⁶ cells/mL and cultured in advanced RPMI medium supplemented with fetal bovine serum, glutamate, HEPES, and recombinant human IL-2. On day 0, cells were stimulated with an equal number of hydrogel complexes conjugated with various ratios of antibodies (anti-CD3, anti-CD27, or anti-CD28 antibodies in the ratios shown in FIG. 8). The numbers of CD4+ and CD8+ T cells in the expanded cell populations are presented as % cell population, and indicate the influence of ligand and ligand density upon the CD4+/CD8+ ratios in expanded cell populations versus day 0 starting population.

### Example 6. Modulation of Ligand and Ligand Density Modulates Memory Phenotypes

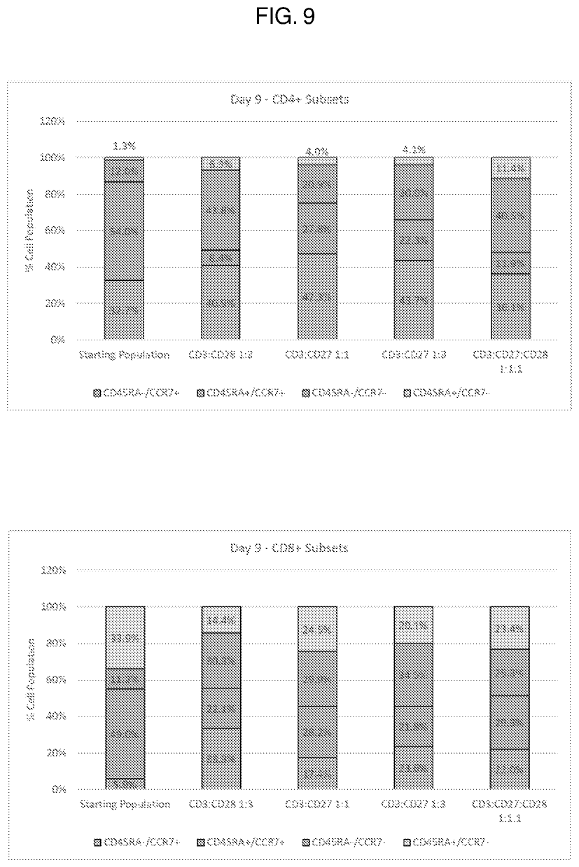

Primary human CD3+T lymphocytes were seeded at a density of 1×10⁶ cells/mL and cultured in advanced RPMI medium supplemented with fetal bovine serum, glutamate, HEPES, and recombinant human IL-2. On day 0, cells were stimulated with an equal number of hydrogel complexes conjugated with various ratios of antibodies (anti-CD3, anti-CD27, or anti-CD28 antibodies in the ratios shown in FIG. 9). CD4+ and CD8+ T cells in the expanded cell populations were analyzed for expression of early T cell memory phenotype markers CD45RA and CCR7, data presented as % cell population, and indicate the influence of ligand and ligand density upon the populations expressing CD45RA and/or CCR7 ratios in expanded CD4+ and CD8+ cell populations versus day 0 starting population.

Other embodiments are in the claims.

What is claimed is:

1. A particle comprising a complex comprising a hydrogel and a binding moiety, wherein:
(a) the hydrogel comprises a polymer; and
(b) the binding moiety is configured to bind a cell surface component of an immune cell.

2. The particle of claim 1, wherein the polymer comprises a natural polymer.

3. The particle of claim 2, wherein the natural polymer is selected from the group consisting of alginate, agarose, carrageenan, chitosan, dextran, carboxymethylcellulose, heparin, hyaluronic acid, polyamino acid, collagen, gelatin, fibrin, a fibrous protein-based biopolymer, and any combination thereof.

4. The particle of claim 1, wherein the polymer comprises a synthetic polymer.

5. The particle of claim 4, wherein the synthetic polymer is selected from the group consisting of alginic acid-polyethylene glycol copolymer, poly(ethylene glycol), poly(2-methyl-2-oxazoline), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ethylhexyl)phthalate, di-iso-nonyl-1,2-cyclohexanedicarboxylate, expanded polytetrafluoroethylene, ethylene vinyl alcohol copolymer, poly(hexamethylene diisocyanate), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), poly(ethylene), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

6. The particle of any one of claims 1-5, wherein the cell surface component is CD2, CD3, CD19, CD24, CD27, CD28, CD31, CD34, CD45, CD46, CD80, CD86, CD133, CD134, CD135, CD137, CD160, CD335, CD337, CD40L, ICOS, GITR, HVEM, Galtectin 9, TIM-1, LFA-1, PD-L1, PD-L2, B7-H3, B7-H4, ILT3, ILT4, CDTL-4, PD-1, BTLA, MHC-I, MHC-II, DLL-Fc, DLL-1, or DLL-4.

7. The particle of any one of claims 1-6, wherein the binding moiety is a cytokine or an antibody or antigen binding fragment thereof.

8. The particle of claim 7, wherein the cytokine is IL-1, IL-2, IL-3, IL-6, IL-7, IL-12, IL-15, IL-18, IL-21, TNF-α, or IFN-γ.

9. The particle of claim 7, wherein the antibody or antigen binding fragment thereof is anti-CD2, anti-CD3, anti-CD19, anti-CD24, anti-CD27, anti-CD28, anti-CD31, anti-CD34, anti-CD45, anti-CD46, anti-CD80, anti-CD86, anti-CD133, anti-CD134, anti-CD135, anti-CD137, anti-CD160, anti-CD335, anti-CD337, anti-CD40L, anti-ICOS, anti-GITR, anti-HVEM, anti-Galtectin 9, anti-TIM-1, anti-LFA-1, anti-PD-L1, anti-PD-L2, anti-B7-H3, anti-B7-H4, anti-ILT3, anti-ILT4, anti-CDTL-4, anti-PD-1, anti-BTLA, anti-MHC-I, anti-MHC-II, anti-DLL-Fc, anti-DLL-1, or anti-DLL-4.

10. The particle of any one of claims 1-6, wherein the binding moiety is chemokine (C-X-C motif) ligand 12 or low-density lipoprotein.

11. The particle of any one of claims 1-10, wherein the immune cell is selected from the group consisting of regulatory T cell, NK cell, NK T cell, CIK cell, TIL cell, HS cell (undifferentiated and differentiated), MS cell (undifferentiated and differentiated), iPS cell (undifferentiated and differentiated), and ES cells (undifferentiated and differentiated).

12. The particle of any one of claims 1-11, wherein the polymer changes from a solid matrix into a solution or suspension in response to a sufficient decrease of cationic concentration in the environment of the polymer.

13. The particle of claim 12, wherein the decrease in the cationic concentration in the environment of the polymer is caused by the presence of EDTA, EGTA, sodium citrate, BAPTA, crown ether, cryptand, phenanthroline sulfonate, dipyridyl sulfonate, dioxane, DME, diglyme, or triglyme.

14. The particle of any one of claims 1-13, wherein the cation is Li⁺, Mg²⁺, Ca²⁺, Sr²⁺, Ba²⁺, Zn²⁺, Cu²⁺, or Al³⁺.

15. The particle of any one of claims 1-14, wherein the hydrogel has an elastic modulus of less than 100,000 pascals (Pa).

16. The particle of any one of claims 1-15, wherein the complex has at least one cross-sectional dimension of between about 1 μm and about 50 μm.

17. The particle of any one of claims 1-16, wherein the complex is substantially spherical and has a diameter of between about 1 μm and 100 μm.

18. The particle of claim 17, wherein the complex has a diameter of between about 5 μm and 15 μm.

19. The particle of any one of claims 1-18, wherein the binding moiety is covalently attached to the hydrogel.

20. The particle of any one of claims 1-19, wherein the binding moiety comprises a signal 1 stimulus.

21. The particle of claim 20, further comprising a signal 2 stimulus.

22. The particle of claim 21, wherein the molar ratio of the signal 1 stimulus and the signal 2 stimulus is between about 1:100 and about 100:1.

23. The particle of claim 22, wherein the molar ratio of the signal 1 stimulus and the signal 2 stimulus is about 1:1.

24. The particle of any one of claims 20-23, wherein the signal 1 stimulus is antigen-specific.

25. The particle of any one of claims 1-24, wherein the binding moiety comprises an antibody or antigen-binding fragment thereof.

26. The particle of claim 25, wherein the antibody or antigen binding fragment thereof is a monoclonal antibody or antigen-binding fragment thereof, a Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′) 2) molecule, a domain antibody, a diabody, a triabody, an affibody, a domain antibody, a SMIP, a nanobody, a Fv fragment, a Fab fragment, a F(ab′) 2 molecule, or a tandem scFv (taFv) fragment.

27. The particle of claim 1, wherein the cell surface component is selected from the group consisting of CD19, CD133, CD135, CD335, CD337, a Delta-like ligand, WNT3, stem cell factor, and thrombopoietin.

28. The particle of claim 20, wherein the signal 1 stimulus is anti-CD3 and/or the signal 2 stimulus is anti-CD28.

29. The particle of any one of claims 1-28, wherein the complex comprises an average of at least one binding moiety per square μm of surface area.

30. The particle of claim 29, wherein the complex comprises an average of at least ten binding moieties per square μm of surface area.

31. A complex comprising a hydrogel and a binding moiety, wherein:
(a) the hydrogel comprises an alginic acid-polyethylene glycol (PEG) copolymer; and
(b) the binding moiety is configured to bind a cell surface component of a T cell.

32. The complex of claim 31, wherein the alginic acid-PEG copolymer changes from a solid matrix into a solution or suspension in response to a sufficient decrease of cationic concentration in the environment of the polymer.

33. The complex of claim 32, wherein the decrease in the cationic concentration in the environment of the polymer is caused by the presence of EDTA, EGTA, sodium citrate, BAPTA, crown ether, cryptand, phenanthroline sulfonate, dipyridyl sulfonate, dioxane, DME, diglyme, or triglyme.

34. The complex of claim 32 or 33, wherein the cation is Li⁺, Mg²⁺, Ca²⁺, Sr²⁺, Ba²⁺, Zn²⁺, Cu²⁺, or Al³⁺.

35. The complex of any one of claims 31-34, wherein the hydrogel has an elastic modulus of less than 100,000 pascals (Pa).

36. The complex of any one of claims 31-35, wherein the alginic acid-PEG copolymer comprises a multi-arm PEG molecule.

37. The complex of claim 36, wherein the multi-arm PEG molecule is a four-arm PEG molecule.

38. The complex of any one of claims 31-37, wherein the complex has at least one cross-sectional dimension of between about 1 μm and about 50 μm.

39. The complex of any one of claims 31-38, wherein the complex is substantially spherical and has a diameter of between about 1 μm and 100 μm.

40. The complex of claim 39, wherein the complex has a diameter of between about 5 μm and 15 μm.

41. The complex of any one of claims 31-40, wherein the binding moiety is covalently attached to the hydrogel.

42. The complex of any one of claims 31-41, wherein the binding moiety comprises a signal 1 stimulus.

43. The complex of claim 42, further comprising a signal 2 stimulus.

44. The complex of claim 43, wherein the molar ratio of the signal 1 stimulus and the signal 2 stimulus is between about 1:100 and about 100:1.

45. The complex of claim 44, wherein the molar ratio of the signal 1 stimulus and the signal 2 stimulus is about 1:1.

46. The complex of any one of claims 42-45, wherein the signal 1 stimulus is antigen-specific.

47. The complex of any one of claims 31-46, wherein the binding moiety comprises an antibody or antigen-binding fragment thereof.

48. The complex of claim 47, wherein the antibody or antigen binding fragment thereof is a monoclonal antibody or antigen-binding fragment thereof, a Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′) 2) molecule, a domain antibody, a diabody, a triabody, an affibody, a domain antibody, a SMIP, a nanobody, a Fv fragment, a Fab fragment, a F(ab′) 2 molecule, or a tandem scFv (taFv) fragment.

49. The complex of claim 47, wherein the binding moiety is selected from the group consisting of anti-CD2, anti-CD3, anti-CD27, anti-CD28, anti-CD46, anti-CD137, and antigen binding fragments thereof.

50. The complex of claim 43, wherein the signal 1 stimulus is anti-CD3 and/or the signal 2 stimulus is anti-CD28.

51. The complex of any one of claims 31-50, wherein the complex comprises an average of at least one binding moiety per square μm of surface area.

52. The complex of claim 51, wherein the complex comprises an average of at least ten binding moieties per square μm of surface area.

53. A complex comprising a hydrogel particle and at least two binding moieties, wherein the hydrogel particle comprises an alginic acid-PEG copolymer and Ca²⁺, and the binding moieties comprises anti-CD3 and anti-CD28, wherein the alginic acid-PEG copolymer changes from a solid matrix into a solution or suspension in response to a sufficient decrease of Ca²⁺ concentration in the environment of the copolymer.

54. The complex of any one of claims 31-53, produced by:
(a) passing an alginic acid-PEG copolymer solution through an atomizer to produce an atomized spray;
(b) contacting the atomized spray with a receiving solution comprising a cation, thereby generating a alginic acid-PEG particle; and
(c) conjugating the binding moieties to the alginic acid-PEG particle to produce the complex.

55. The complex of claim 54, wherein the alginic acid-PEG copolymer solution flows through the atomizer at a volumetric percentage from 30% to 90%.

56. The complex of claim 54 or 55, wherein the atomizer is injected with a gas at a pressure from 1 to 200 pounds per square inch (psi).

57. The complex of any one of claims 54-56, wherein the alginic acid-PEG copolymer solution is injected into the atomizer at a flow rate from 0.1 to 100 mL per minute.

58. The complex of any one of claims 54-57, wherein the atomizer is an external mix atomizer.

59. The complex of any one of claims 54-58, wherein the atomizer produces a round spray pattern.

60. The complex of any one of claims 54-59, wherein the atomizer produces a spray angle from 10° to 30°.

61. A method of producing an alginic acid-PEG particle, the method comprising:
(a) passing an alginic acid-PEG copolymer solution through an atomizer to produce an atomized solution; and
(b) contacting the atomized solution with a receiving solution comprising a cation, thereby generating an alginic acid-PEG particle.

62. The method of claim 61, further comprising conjugating a binding moiety to the alginic acid-PEG particle to produce a hydrogel complex.

63. The method of claim 62, wherein the hydrogel complex has an elastic modulus of less than 100,000 Pa.

64. The method of any one of claims 61-63, wherein the binding moiety binds to a surface component of a T cell.

65. The method of any one of claims 61-64, wherein the alginic acid-PEG copolymer solution flows through the atomizer at a volumetric percentage from 30% to 90%.

66. The method of any one of claims 61-65, wherein the atomizer is injected with a gas at a pressure from 1 to 200 psi.

67. The method of any one of claims 61-66, wherein the alginic acid-PEG copolymer solution is injected into the atomizer at a flow rate from 0.1 to 100 mL per minute.

68. The method of any one of claims 61-67, wherein the atomizer is an external mix atomizer.

69. The method of any one of claims 61-68, wherein the atomizer produces a round spray pattern.

70. The method of any one of claims 61-69, wherein the atomizer produces a spray angle from 10° to 30°.

71. A method of generating a population of expanded T cells, the method comprising contacting a starting population of T cells with a plurality of complexes, each complex comprising a hydrogel and a binding moiety, wherein:
(i) the hydrogel comprises an alginic acid-PEG copolymer; and
(ii) the binding moiety binds a cell surface component of a T cell;
and wherein the contact is operative to induce a metabolic change in the starting population of T cells, thereby generating a population of expanded T cells.

72. The method of claim 71, wherein the complex changes from a solid matrix into a solution or suspension in response to a sufficient decrease of cationic concentration in the environment of the polymer.

73. The method of claim 71 or 72, wherein the complexes are administered to a culture comprising the population of T cells at a complex-to-cell ratio from 1:1 to 20:1.

74. The method of claim 73, wherein the complex-to-cell ratio is a complex-to-T cell ratio.

75. The method of claim 74, wherein the complex-to-T cell ratio is about 5:1.

76. The method of claim 73, wherein the complex-to-cell ratio is a complex-to-peripheral blood mononuclear cell (PBMC) ratio.

77. The method of claim 76, wherein the complex-to-PBMC ratio is about 10:1.

78. The method of any one of claims 71-77, wherein the population of expanded T cells comprises a greater number or percentage of CD8⁺ T cells than the starting population.

79. The method of any one of claims 71-78, wherein the population of expanded T cells comprises a lower number or percentage of CD4⁺ T cells than the starting population.

80. The method of any one of claims 71-79, wherein the population of expanded T cells comprises a greater CD8-to-CD4 T cell ratio than the starting population.

81. The method of any one of claims 71-80, wherein the population of expanded T cells comprises 100-fold the number of T cells relative to the starting population.

82. The method of any one of claims 71-81, wherein the population of expanded T cells comprises activated T cells.

83. The method of any one of claims 61-82, wherein the hydrogel has an elastic modulus of less than 100,000 pascals (Pa).

84. The method of any one of claims 61-83, wherein the alginic acid-PEG copolymer comprises a multi-arm PEG molecule.

85. The method of claim 84, wherein the multi-arm PEG molecule is a four-arm PEG molecule.

86. The method of any one of claims 61-85, wherein the complex has at least one cross-sectional dimension of between about 1 μm and about 50 μm.

87. The method of any one of claims 61-86, wherein the complex is substantially spherical and has a diameter of between about 1 μm and 100 μm.

88. The method of claim 87, wherein the complex has a diameter of between about 5 μm and 15 μm.

89. The method of any one of claims 61-88, wherein the binding moiety is covalently attached to the hydrogel.

90. The method of any one of claims 61-89, wherein the binding moiety comprises a signal 1 stimulus.

91. The method of claim 90, wherein the complex further comprises a signal 2 stimulus.

92. The method of claim 91, wherein the molar ratio of the signal 1 stimulus and the signal 2 stimulus is between about 1:100 and about 100:1.

93. The method of claim 92, wherein the molar ratio of the signal 1 stimulus and the signal 2 stimulus is about 1:1.

94. The method of any one of claims 90-93, wherein the signal 1 stimulus is antigen-specific.

95. The method of any one of claims 61-94, wherein the binding moiety comprises an antibody or antigen-binding fragment thereof.

96. The method of claim 95, wherein the antibody or antigen binding fragment thereof is a monoclonal antibody or antigen-binding fragment thereof, a Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′) 2) molecule, a domain antibody, a diabody, a triabody, an affibody, a domain antibody, a SMIP, a nanobody, a Fv fragment, a Fab fragment, a F(ab′) 2 molecule, or a tandem scFv (taFv) fragment.

97. The method of claim 96, wherein the binding moiety is selected from the group consisting of anti-CD2, anti-CD3, anti-CD27, anti-CD28, anti-CD46, anti-CD137, and antigen binding fragments thereof.

98. The method of claim 91, wherein the signal 1 stimulus is anti-CD3 and/or the signal 2 stimulus is anti-CD28.

99. The method of any one of claims 61-98, wherein the complex comprises an average of at least one binding moiety per square μm of surface area.

100. The method of claim 99, wherein the complex comprises an average of at least ten binding moieties per square μm of surface area.

101. A method of generating a population of expanded immune cells, the method comprising contacting a starting population of immune cells with a plurality of particles of any one of claims 1-30;
wherein the contact is operative to induce a metabolic change in the starting population of immune cells, thereby generating a population of expanded immune cells.

102. The method of claim 101, wherein the particles change from a solid matrix into a solution or suspension in response to a sufficient decrease of cationic concentration in the environment of the polymer.

103. The method of claim 101 or 102, wherein the particles are administered to a culture comprising the population of immune cells at a particle-to-cell ratio from 1:20 to 20:1.

104. The method of claim 103, wherein the particle-to-cell ratio is a particle-to-immune cell ratio.

105. The method of claim 104, wherein the particle-to-immune cell ratio is about 5:1.

106. The method of claim 103, wherein the particle-to-cell ratio is a complex-to-peripheral blood mononuclear cell (PBMC) ratio.

107. The method of claim 106, wherein the particle-to-PBMC ratio is about 10:1.

108. The method of any one of claims 101-107, wherein the population of expanded immune cells comprises 100-fold the number of immune cells relative to the starting population.

109. The method of any one of claims 101-108, wherein the population of expanded immune cells comprises activated immune cells.
